(19) United States
(12) Patent Application Publication
(10) Pub. No.: US 2026/0177525 A1
(43) Pub. Date: Jun. 25, 2026

(54) NANOPORE FLUIDIC DEVICE AND METHODS OF MAKING THE SAME

(71) Applicant: ILLUMINA, INC., San Diego, CA (US)

(72) Inventors: Blake Bloomfield, San Diego, CA (US); Arjun Nair, San Diego, CA (US); Mario Vazquez Garcia, Chula Vista, CA (US); Sharis Minassian, San Marcos, CA (US)

(21) Appl. No.: 19/423,895

(22) Filed: Dec. 17, 2025

Related U.S. Application Data
(60) Provisional application No. 63/736,442, filed on Dec. 19, 2024.

(51) Int. Cl. G01N 27/447; C12Q 1/6869; G03F 7/00; G03F 7/16; G03F 7/20

(52) U.S. Cl. CPC G01N 27/44791; C12Q 1/6869; G03F 7/0015; G03F 7/168; G03F 7/2004; G03F 7/70008

(57) ABSTRACT

A nanopore fluidic device includes a support structure having a top hydrophobic surface and having hydrophilic sidewalls defining a plurality of trans wells; a trans electrode associated with each of the plurality of trans wells; one or more cis wells facing the plurality of trans wells; and a cis electrode associated with each of the one or more cis wells.

### CROSS-REFERENCE TO RELATED APPLICATION

This application claims the benefit of U.S. Provisional Application Ser. No. 63/736,442, filed Dec. 19, 2024, the content of which is incorporated by reference herein in its entirety.

### BACKGROUND

Time and energy have been invested in exploring the use of nanopores for a variety of applications, including polynucleotide sequencing, protein identification and/or quantification, analyte detection, etc. However, some devices, systems, and methods that utilize nanopores still lack robustness, reproducibility, and/or sensitivity. These devices, systems, and methods may not have sufficiently high throughput for practical implementation, such as in demanding commercial applications (e.g., genome sequencing in clinical and other settings), that demand cost effective and highly accurate operation.

### SUMMARY

The fluidic devices disclosed herein include a cis well and a plurality of trans wells that are separated from the cis well by respective membranes. Each trans well is in fluid communication with the cis well by nanopores inserted in the respective membranes. The fluidic device includes a support structure, which has a top hydrophobic surface and hydrophilic sidewalls that define a plurality of the trans wells. The top hydrophobic surface helps to ensure that the membranes stay in place. For example, the top hydrophobic surface captures and retains oil while repelling moisture, which contribute to membrane retention. The hydrophilic sidewalls of the plurality of the trans wells help to capture and retain electrolyte buffer solutions.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a schematic illustration of one example of a nanopore fluidic device;

FIG. 1B is a schematic illustration of another example of a nanopore fluidic device;

FIG. 2 schematically illustrates a single trans well with a membrane formed in its aperture, where the membrane has a bilayer structure and a nanopore inserted therein;

FIG. 3A through FIG. 3C together illustrate one example of the method disclosed herein;

FIG. 4A through FIG. 4C together illustrate another example of the method disclosed herein;

FIG. 5A through FIG. 5E together illustrate another example of the method disclosed herein;

FIG. 6A through FIG. 6C together illustrate another example of the method disclosed herein;

FIG. 7A through FIG. 7E together illustrate another example of the method disclosed herein;

FIG. 8 is a graph depicting the contact angle (°, Y axis) of four support structures formed via the method of FIG. 7A through FIG. 7E at different times throughout the process (identified on the X axis);

FIG. 9A is a graph depicting the Nernst behavior (i.e., potential (vs. Ag/AgCl, V, Y axis)) versus the log[Cl−] (X axis) of Ag/AgCl electrodes before and after SF₆ exposure; and

FIG. 9B is a graph depicting open circuit potential (OCP (vs. Ag/AgCl, V, Y axis) versus time (hours, X axis) for Ag/AgCl electrodes before and after SF₆ exposure.

### DETAILED DESCRIPTION

Fluidic devices and nanopore instruments are described herein that exhibit improved membrane and buffer solution retention. The fluidic devices include a support structure that is configured with both a hydrophobic top surface and hydrophilic sidewalls. The improved membrane retention is due to the hydrophobic top surface, and the improved buffer solution retention is due to the hydrophilic sidewalls.

### Definitions

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” is also intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine, or the like. Certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

As used herein, a “polymerase” is intended to mean an enzyme having an active site that assembles polynucleotides by polymerizing nucleotides into polynucleotides. A polymerase can bind a primer and a single stranded target polynucleotide and sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP. Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, DEEP VENTR™ (exo-) DNA polymerase, DEEP VENTR™ DNA polymerase, DYNAZYME™ EXT DNA, DYNAZYME™ II Hot Start DNA Polymerase, PHUSION™ High-Fidelity DNA Polymerase, THERMINATOR™ DNA Polymerase, THERMINATOR™ II DNA Polymerase, VENTR® DNA Polymerase, VENTR® (exo-) DNA Polymerase, REPLIPHI™ Phi29 DNA Polymerase, rBst DNA Polymerase, rBst DNA Polymerase (Large), Fragment (ISOTHERM™ DNA Polymerase), MASTERAMP™ AMPLITHERM™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and ISOPOL™ SD+ polymerase. In specific examples, the polymerase is selected from a group consisting of Bst, Bsu, and Phi29. Some polymerases have an activity that degrades the strand behind them (3′ exonuclease activity). Some useful polymerases have been modified, either by mutation or otherwise, to reduce or eliminate 3′ and/or 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NS5B protein. Example RNA Reverse Transcriptases include those derived from Avian Myelomatosis Virus (AMV), Murine Moloney Leukemia Virus (MMLV) and/or the Human Immunodeficiency Virus (HIV), telomerase reverse transcriptases such as (hTERT), SUPERSCRIPT™ III, SUPERSCRIPT™ IV Reverse Transcriptase, and PROTOSCRIPT® II Reverse Transcriptase.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-guanine, which may be cleaved under suitable conditions, such as UV light, or upon exposure to chemistry, an enzyme, or another suitable cleaving mechanism. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more of the same or different members. Pluralities may range in size from small, medium, large, to very large. The size of a small plurality may range, for example, from a few members to tens of members. Medium sized pluralities may range, for example, from about tens of members to about hundreds of members. Large pluralities may range, for example, from about hundreds of members to about thousands of members, or up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Accordingly, the definition of the term is intended to include all integer values greater than two.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to sample preparation, sequencing, and/or another procedure. A target polynucleotide may include nucleotide sequences additional to a target sequence to be analyzed. For example, a target polynucleotide may include one or more adapters, including (an) amplification adapter(s) that function(s) as (a) primer binding site(s), that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such an adapter may be located at either the 3′ end or the 5′ end the target polynucleotide. Target polynucleotides may be used without any adapter, in which case a primer binding sequence may come directly from a sequence found in the target polynucleotide.

As used herein, the term “support structure” refers to a material or a combination of materials used as a foundation for supporting composition(s) (e.g., the membrane) described herein. The support structure may be defined out of a single material, or may include a substrate upon which other components (e.g., a patterned layer) of the support structure are positioned.

Example substrate materials may include glass, silica, plastic, quartz, metal, metal oxide, organo-silicate (e.g., polyhedral organic silsesquioxanes (commercially available under the tradename POSS® from Hybrid Plastics)), polyacrylates, tantalum oxide, complementary metal oxide semiconductor (CMOS), or combinations thereof. An example of POSS® can be that described in Kehagias et al., Microelectronic Engineering 86 (2009), pp. 776-778, which is incorporated by reference in its entirety. In some examples, the substrate is a silica-based substrate, such as glass, fused silica, or other silica-containing material. In some examples, silica-based substrates can include silicon, silicon dioxide, silicon nitride, or silicone hydride. In other examples, the substrate is a polymer or includes a polymeric component, such as polyethylene, polystyrene, poly(vinyl chloride), polypropylene, polyamides (i.e., nylons), polyimide, polyesters, polycarbonates, and poly(methyl methacrylate). Example polymeric materials include poly(methyl methacrylate), polystyrene, and cyclic olefin polymer substrates. In some examples, the substrate is or includes a silica-based material or a polymeric material or a combination thereof. In particular examples, the substrate has at least one surface including glass or a silicon-based polymer. In some examples, the substrate can include a metal, such as gold. In some examples, the substrate has at least one surface including a metal oxide. In one example, the surface includes a tantalum oxide or tin oxide. Acrylamides, enones, or acrylates may also be utilized as a substrate material or component. Other example substrate materials include gallium arsenide (GaAs), indium tin oxide (ITO), indium phosphide, aluminum, ceramics, quartz, resins, or other polymers and copolymers. In some examples, the substrate and/or the substrate surface can be, or include, quartz. In some other examples, the substrate and/or the substrate surface can be, or include, a semiconductor, such as GaAs or ITO. The foregoing lists are intended to be illustrative, but not limiting, of the present application. Substrates can include a single material or a plurality of different materials. Substrates can be composites or laminates. In some examples, the substrate includes an organo-silicate material.

The patterned layer of the support structure may be of biological origin, or may be synthetic. As some examples, the patterned layer may include or may consist essentially of: an organic material, e.g., a curable resin such as SU-8; polytetrafluoroethylene (PTFE), poly(methyl methacrylate) (PMMA), parylene, or the like. Additionally, or alternatively, in various examples, the patterned layer may include or may consist essentially of an inorganic material, e.g., silicon nitride, silicon oxide, or molybdenum disulfide.

The support structure can suspend a membrane. The support structure may also define an aperture, such that a first portion of the membrane is suspended across the aperture, and a second portion of the membrane is disposed on, and supported by, the support structure. The support structure may include any suitable arrangement of elements to define the aperture and suspend the membrane across the aperture. As mentioned, the support structure may include the patterned layer, which may have the aperture defined therein, which opens to a well that is also defined therein. Additionally, or alternatively, the patterned layer of the support structure may include one or more first features (such as one or more lips or ledges of the well) that are raised relative to one or more second features (such as a bottom surface of the well), wherein a height difference between (a) the one or more first features and (b) the one or more second features defines the well and the aperture across which the membrane may be suspended. The aperture may have any suitable shape, such as a circle, an oval, a polygon, or an irregular shape.

In some examples, the support structure described herein forms at least part of a fluidic device, or is located in or coupled to a fluidic device. One example of a fluidic device is a flow cell including a single flow channel (e.g., a single cis well in fluid communication with a plurality of trans wells). Another example of a fluidic device is a flow cell including multiple flow channels (e.g., separate cis wells that are in respective fluid communication with separate sets of trans wells). Example flow cells and support structures for the manufacture of flow cells that can be used in methods and compositions set forth herein include, but are not limited to, those commercially available from Illumina, Inc. (San Diego, CA).

As used herein, the term “electrode” is intended to mean a solid structure that conducts electricity. Electrodes may include any suitable electrically conductive material, such as gold, palladium, silver, silver/silver chloride, platinum, or combinations thereof. In some examples, an electrode may be disposed on a substrate. In some examples, an electrode may be a component of a support structure.

As used herein, the term “nanopore” is intended to mean a structure that includes an aperture that permits molecules to cross therethrough from a first side of the nanopore to a second side of the nanopore, in which a portion of the aperture of a nanopore has a width of 100 nm or less, e.g., 10 nm or less, or 2 nm or less. The aperture extends through the first and second sides of the nanopore. Molecules that can cross through an aperture of a nanopore can include, for example, ions or water-soluble molecules, such as amino acids or nucleotides. The nanopore can be disposed within a barrier, or can be provided through a substrate. Optionally, a portion of the aperture can be narrower than one or both of the first and second sides of the nanopore, in which case that portion of the aperture can be referred to as a “constriction.” Alternatively or additionally, the aperture of a nanopore, or the constriction of a nanopore (if present), or both, can be greater than 0.1 nm, 0.5 nm, 1 nm, 10 nm or more. A nanopore can include multiple constrictions, e.g., at least two, or three, or four, or five, or more than five constrictions. Nanopores include biological nanopores or hybrid nanopores.

Biological nanopores include, for example, polypeptide nanopores and polynucleotide nanopores. A “polypeptide nanopore” is intended to mean a nanopore that is made from one or more polypeptides. The one or more polypeptides can include a monomer, a homopolymer, or a heteropolymer. Structures of polypeptide nanopores include, for example, an α-helix bundle nanopore and a β-barrel nanopore as well as all others well known in the art. Example polypeptide nanopores include aerolysin, α-hemolysin, Mycobacterium smegmatis porin A (MspA), gramicidin A, maltoporin, OmpF, OmpC, PhoE, Tsx, F-pilus, SP1, mitochondrial porin (VDAC), Tom40, outer membrane phospholipase A, CsgG, CsgG-CsgF complex, and Neisseria autotransporter lipoprotein (NaIP). Mycobacterium smegmatis porin A (MspA) is a membrane porin produced by Mycobacteria, allowing hydrophilic molecules to enter the bacterium. MspA forms a tightly interconnected octamer and transmembrane beta-barrel that resembles a goblet and includes a central constriction. For further details regarding α-hemolysin, see U.S. Pat. No. 6,015,714, the entire contents of which are incorporated by reference herein. For further details regarding SP1, see Wang et al., Chem. Commun., 49:1741-1743 (2013), the entire contents of which are incorporated by reference herein. For further details regarding MspA, see Butler et al., “Single-molecule DNA detection with an engineered MspA protein nanopore,” Proc. Natl. Acad. Sci. 105:20647-20652 (2008) and Derrington et al., “Nanopore DNA sequencing with MspA,” Proc. Natl. Acad. Sci. USA, 107:16060-16065 (2010), the entire contents of both of which are incorporated by reference herein. Other nanopores include, for example, the MspA homolog from Nocardia farcinica, and lysenin. For further details regarding lysenin, see PCT Publication No. WO 2013/153359, the entire contents of which are incorporated by reference herein. A “polynucleotide nanopore” is intended to mean a nanopore that is made from one or more nucleic acid polymers. A polynucleotide nanopore can include, for example, a polynucleotide origami.

A “hybrid nanopore” is intended to mean a nanopore that is made from two different materials of biological origin or from materials of both biological and non-biological origins. Materials of biological origin are defined above and include, for example, polypeptides and polynucleotides. One example of a hybrid nanopore that includes two different materials of biological origin is a polynucleotide origami-protein nanopore complex. As mentioned, the hybrid nanopore may include materials of both biological and non-biological origins. Solid-state nanopores are one example of a nanopore that is made from one or more materials that are not of biological origin. Examples of solid-state nanopores include silicon nitride (SiN), silicon dioxide (SiO₂), silicon carbide (SiC), hafnium oxide (HfO₂), molybdenum disulfide (MoS₂), hexagonal boron nitride (h-BN), or graphene). Examples of biological and solid-state hybrid nanopores include a polypeptide-solid-state hybrid nanopore and a polynucleotide-solid-state nanopore.

As used herein, the term “nanopore subunits” refers to the units that self-assemble to form some biological nanopores. The units may be protein units or polypeptide units. Example protein nanopores that are composed of multiple protein subunits include MspA, CsgG, CsgG/CsgF, Fragacea-toxin C, Aerolysin, and aHL.

As used herein, a “membrane” is intended to mean a structure that normally inhibits passage of molecules from one side of the membrane to the other side of the membrane. The molecules for which passage is inhibited can include, for example, ions or water-soluble molecules, such as nucleotides and amino acids. However, if a nanopore is disposed within a membrane, then the aperture of the nanopore may permit passage of molecules from one side of the membrane to the other side of the membrane. As one specific example, if a nanopore is disposed within a membrane, the aperture of the nanopore may permit passage of molecules from one side of the membrane to the other side of the membrane. Membranes suitable for use in the examples disclosed herein include amphiphilic bilayers, including those of biological origin, such as amphiphilic lipid bilayers, or those of synthetic origin, such as amphiphilic polymer bilayers.

As used herein, “of biological origin” refers to material derived from or isolated from a biological environment such as an organism or cell, or a synthetically manufactured version of a biologically available structure.

As used herein, “synthetic” refers to a membrane material that is not of biological origin (e.g., polymeric materials, synthetic phospholipids, or combinations thereof).

As used herein, a “solution” is intended to refer to a homogeneous mixture including two or more substances. In such a mixture, a solute is a substance which is dissolved in another substance referred to as a solvent. A solution may include a single solute, or may include a plurality of solutes. An “aqueous solution” refers to a solution in which the solvent is, or includes, water.

As used herein, a “polymeric membrane” or a “polymer membrane” refers to a synthetic barrier that primarily is composed of a polymer that is not of biological origin. In some examples, a polymeric membrane consists essentially of a polymer that is not of biological origin. An amphiphilic block copolymer is an example of a polymer that is not of biological origin and that may be included in the present membranes. A hydrophobic polymer with ionic end groups is another example of a polymer that is not of biological origin and that may be included in the present membranes.

As used herein, the term “block copolymer” is intended to refer to a polymer having at least a first portion or “block” that includes a first type of monomer, and at least a second portion or “block” that is coupled directly or indirectly to the first portion and includes a second, different type of monomer. The first portion may include a polymer of the first type of monomer, or the second portion may include a polymer of the second type of monomer, or the first portion may include a polymer of the first type of monomer and the second portion may include a polymer of the second type of monomer. In the examples set forth herein, at least one of the blocks is hydrophilic and at least one other of the blocks is hydrophobic. In some examples, the monomers themselves impart the desired hydrophilicity and hydrophobicity. In other examples, the first portion optionally may include an end group with a hydrophilicity that is different than that of the first type of monomer and/or the second portion optionally may include an end group with a hydrophilicity that is different than that of the second type of monomer. The end groups of any hydrophilic blocks may be located at an outer surface of a barrier/membrane formed using such hydrophilic blocks. Depending on the particular configuration, the end groups of any hydrophobic blocks may be located at an inner surface of the barrier or at an outer surface of a barrier formed using such hydrophobic blocks.

Block copolymers include, but are not limited to, diblock copolymers and triblock copolymers.

A “diblock copolymer” is intended to refer to a block copolymer that includes, or consists essentially of, first and second blocks coupled directly or indirectly to one another. The first block may be hydrophilic, and the second block may be hydrophobic, in which case the diblock copolymer may be referred to as an “AB” copolymer where “A” refers to the hydrophilic block and “B” refers to the hydrophobic block.

A “triblock copolymer” is intended to refer to a block copolymer that includes, or consists essentially of, first, second, and third blocks coupled directly or indirectly to one another. The first and third blocks may include, or may consist essentially of, the same type of monomer (repeating unit) as one another, and the second block may include a different type of monomer (repeating unit). In other examples, the first block may be hydrophilic, the second block may be hydrophobic, and the third block may be hydrophilic and include the same type of monomer as the first block, in which case the triblock copolymer may be referred to as an “ABA” copolymer where “A” refers to the hydrophilic blocks and “B” refers to the hydrophobic block.

The particular arrangement of molecules of polymer chains (e.g., block copolymers) within a polymeric membrane may depend, among other things, on the respective block lengths, the type(s) of monomers used in the different blocks, the relative hydrophilicity and hydrophobicity of the blocks, the composition of the fluid(s) within which the membrane is formed, and/or the density of the polymeric chains within the membrane. During formation of the membrane, these and other factors generate forces between molecules of the polymeric chains which laterally position and reorient the molecules in such a manner as to substantially minimize the free energy of the membrane. The membrane may be considered to be substantially “stable” once the polymeric chains have completed these rearrangements, even though the molecules may retain some fluidity of movement within the membrane.

As used herein, the term “hydrophobic” is intended to mean tending to exclude water molecules. Hydrophobicity is a relative concept relating to the polarity difference of molecules relative to their environment. Non-polar (hydrophobic) molecules in a polar environment will tend to associate with one another in such a manner as to reduce contact with polar (hydrophilic) molecules to a minimum to lower the free energy of the system as a whole.

As used herein, the term “hydrophilic” is intended to mean tending to bond to water molecules. Polar (hydrophilic) molecules in a polar environment will tend to associate with one another in such a manner as to reduce contact with non-polar (hydrophobic) molecules to a minimum to lower the free energy of the system as a whole.

As used herein, the term “amphiphilic” is intended to mean having both hydrophilic and hydrophobic properties. For example, a block copolymer that includes a hydrophobic block and a hydrophilic block may be considered amphiphilic. Illustratively, AB copolymers and ABA copolymers all may be considered amphiphilic.

As used herein, the term “linker” is intended to mean a moiety, molecule, or molecules via which one element is attached to another element. Linkers may be covalent or may be non-covalent. Examples of covalent linkers include moieties such as alkyl chains, polyethers, amides, esters, aryl groups, polyaryls, and the like. Examples of noncovalent linkers include host-guest complexation, cyclodextrin/norbornene, adamantane inclusion complexation with β-CD, DNA hybridization interactions, streptavidin/biotin, and the like. In examples of the block copolymers set forth herein, the linker is covalent.

As used herein, the terms “PEO”, “PEG”, “poly(ethylene oxide)”, and “poly(ethylene glycol)” are intended to be used interchangeably and refer to a polymer that comprises —[CH₂—CH₂—O]ₙ—. In some examples, n is between about 2 and about 100.

As used herein, the term “annulus” is intended to refer to a liquid that is adhered to a support structure, that is located within a membrane, that extends partially into the aperture, and that is well defined by the support structure. As such, it will be understood that the annulus may follow the shape of the membrane, e.g., may have the shape of a circle, an oval, a polygon, or an irregular shape.

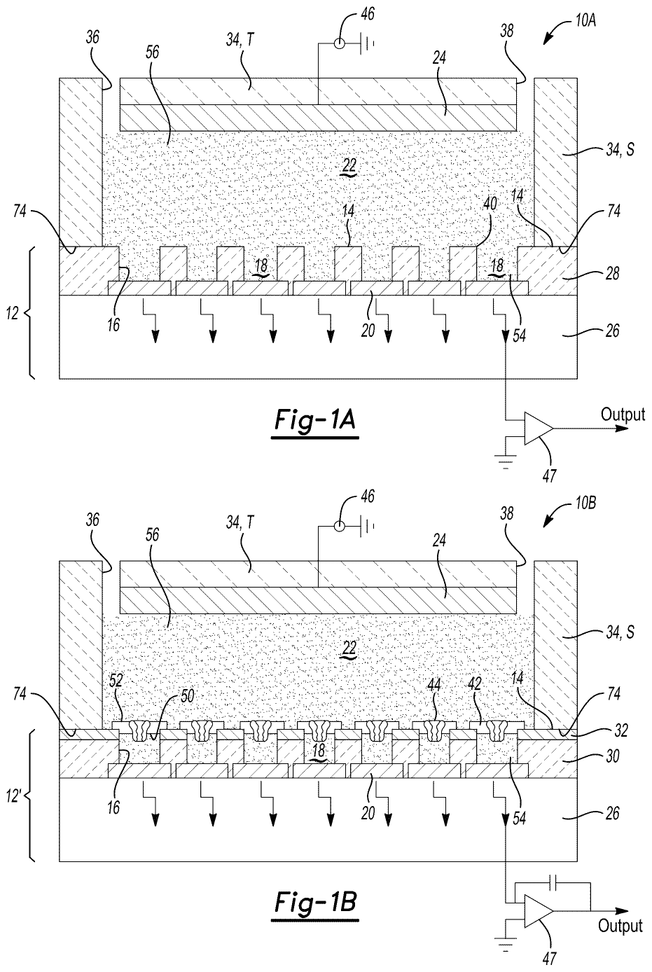

The term “operable position” refers to the configuration of the membrane and nanopore when the fluidic device or the nanopore instrument is to be used in a sequencing operation, analyte capture, proteomics, DNA profiling, spatial surveillance, minimal residual disease (MRD) surveillance, or pathogen surveillance. In the examples set forth herein, the operable position of the membrane is when it is suspended over the aperture of a well, and the operable position of the nanopore is when it is inserted into the membrane. The operable position of the membrane and nanopore are depicted in FIG. 1B.

The term “inoperable position” refers to the configuration of the membrane and nanopore when the fluidic device or the nanopore instrument is to be used in pre-operational processes, such as handling, shipping and/or storage. In the examples set forth herein, the inoperable position of the membrane is when it is positioned over the interstitial regions, and the inoperable position of the nanopore is when it is attached to the membrane over the interstitial regions.

As used herein, a “negative photoresist” refers to a light sensitive material in which a portion that is exposed to light of a particular wavelength(s) becomes insoluble to a developer. In these examples, the insoluble negative photoresist has less than 5% solubility in the developer. With the negative photoresist, the light exposure changes the chemical structure so that exposed portions of the material become less soluble (than non-exposed portions) in the developer. While not soluble in the developer, the insoluble negative photoresist may be at least 99% soluble in a remover that is different from the developer. The remover may be a solvent or solvent mixture used, e.g., in a lift-off process.

In contrast to the insoluble negative photoresist, any portion of the negative photoresist that is not exposed to light is at least 95% soluble in the developer. This portion may be referred to as a “soluble negative photoresist”. In some examples, the soluble negative photoresist is at least 98%, e.g., 99%, 99.5%, 100%, soluble in the developer.

### Nanopore Fluidic Devices

Different example nanopore fluidic devices 10A, 10B are shown in FIG. 1A and FIG. 1B. Each device 10A, 10B includes a support structure 12, 12′ having a top hydrophobic surface 14 and having hydrophilic sidewalls 16 defining a plurality of trans wells 18; a trans electrode 20 associated with each of the plurality of trans wells 18; one or more cis wells 22 facing the plurality of trans wells 18; and a cis electrode 24 associated with each of the one or more cis wells 22.

In the example shown in FIG. 1A, the support structure 12 includes a substrate 26 and a patterned layer 28 thereon, where the patterned layer 28 defines both the top hydrophobic surface 14 and the hydrophilic sidewalls 16.

The substrate 26 may be any of the examples set forth herein in the definitions. In one example, the substrate 26 is a semiconductor (e.g., silicon) wafer.

The patterned layer 28 may be deposited over the substrate 26 by any suitable technique, such as spin coating, chemical vapor deposition, dip coating, dunk coating, spray coating, puddle dispensing, ultrasonic spray coating, doctor blade coating, aerosol printing, screen printing, etc. The features formed in the patterned layer 28 may be formed by any suitable technique, such as photolithography, imprint lithography, microcontact printing, and other suitable techniques.

In one embodiment, the patterned layer 28 may be any negative photoresist patterned by photolithography whose surface is susceptible to a decrease in surface energy when exposed to a fluorine-based plasma, such as sulfur hexafluoride (SF₆), tetrafluoromethane (CF₄), or nitrogen trifluoride (NF₃), or to a non-fluorine-based plasma, such as tetramethyl silane (Si(CH₃)₄). The bulk of the negative photoresist, however, is a high-surface energy material. An example of a suitable negative photoresist includes the NR® series photoresist (available from Futurrex). Other suitable negative photoresists include some resists of the SU-8 Series (e.g., SU-8 2000 or 3000) and the KMPR® Series (both of which include epoxy-based photoresists available from Kayaku Advanced Materials, Inc.), or SUEX® (an epoxy photoresist from DJ Microlaminates, Inc.), or SINR (siloxane-based photoresist), or TMMF (dry film photoresists from Tokyo Ohka Kogyo (TOK)). The patterned layer 28, including the top hydrophobic surface 14 and the hydrophilic sidewalls 16, may be formed as described in reference to FIG. 3A through FIG. 3C.

In this example, the top hydrophobic surface 14 comprises a low surface energy plasma treatment. As examples, the low surface energy plasma treatment comprises a fluorine plasma treatment; or the low surface energy plasma treatment comprises a tetramethyl silane.

The material selected for the patterned layer 28 is also capable of being processed with nanometer or micrometer accuracy for defining the trans wells 18.

The trans wells 18 may be micro wells (having at least one dimension on the micron scale, e.g., about 1 μm up to, but not including, 1000 μm), such as from about 1 μm to about 750 μm, such as from about 5 μm to about 500 μm, such as from about 10 μm to about 250 μm, such as from about 15 μm to about 100 μm. Each trans well 18 may be characterized by its aspect ratio (e.g., depth or height divided by width or diameter). In an example, the aspect ratio of each trans well 18 may range from about 1:2 to about 10:1. In another example, the aspect ratio of each trans well 18 may range from about 1:1 to about 5:1.

When a plurality of the trans wells 18 are used, many different layouts of multiple trans wells 18 may be envisaged, including regular, repeating, and non-regular patterns. In an example, the trans wells 18 are disposed in a hexagonal grid for close packing and improved density. Other layouts may include, for example, rectangular layouts, triangular layouts, and so forth. As examples, the layout or pattern can be an x-y format of trans wells 18 that are in rows and columns.

The layout may be characterized with respect to the number of the trans wells 18 per substrate or die. For one example, the trans wells 18 may be present at over 1,000 wells or pixels per substrate or die. For another example, the trans wells 18 may be present at over 10,000 wells or pixels per substrate or die. For yet another example, the trans wells 18 may be present at over 100,000 wells or pixels per substrate or die. For still another example, the trans wells 18 may be present at over 500,000 wells or pixels per substrate or die. For a further example, the trans wells 18 may be present at over 1,000,000 wells or pixels per substrate or die.

The layout may also or alternatively be characterized in terms of the average pitch, i.e., the spacing from the center of a trans well 18 to the center of an adjacent trans well 18 (center-to-center spacing). The pattern can be regular such that the coefficient of variation around the average pitch is small, or the pattern can be non-regular in which case the coefficient of variation can be relatively large. In an example, the average pitch may range from about 1 μm to about 1,000 μm. The average pitch can be, for example, at least about 1 μm or at least about 10 μm. Alternatively or additionally, the average pitch can be, for example, at most 1,000 μm, about 500 μm, about 100 μm, about 50 μm, about 20 μm, or less. The average pitch for an example array including a particular pattern of trans wells 18 can be between one of the lower values and one of the upper values selected from the ranges above.

In the example shown in FIG. 1A, each trans well 18 includes the hydrophilic sidewalls 16, which are defined by the patterned layer 28.

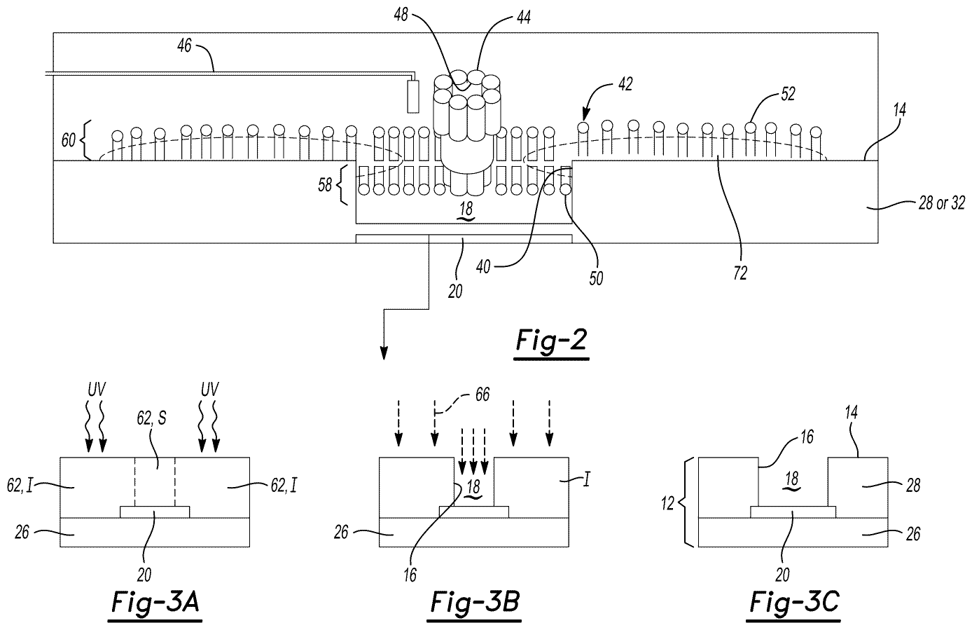

Each of the trans well(s) 18 also has an aperture 40. The aperture 40 is the opening into the well 18. The aperture 40 faces the cis well 22, and, as shown in FIG. 1B, is large enough to accommodate at least a portion of a membrane 42 and a nanopore 44 that is associated therewith when these components are in the operable position. For example, an end of the nanopore 44 may extend through the membrane 42 and into the aperture 40 of the trans well 18 (as shown in FIG. 1B and FIG. 2).

The lower surface of each trans well 18 is defined by the trans electrode 20. More specifically, respective trans electrodes 20 are positioned on the substrate 26 at the bottom of each trans well 18. As such, a surface of the trans electrode 20 defines the lower surface of the trans well 18. In the example shown in FIG. 1A, the trans electrodes 20 extend beyond the hydrophilic sidewalls 16 such that the patterned layer 28 is positioned over the perimeter of each trans electrode 20.

The trans electrodes 20 may be any suitable electrode material, such as gold (Au), platinum (Pt), ruthenium (Ru), carbon (C) (e.g., graphite, diamond, etc.), silver/silver chloride (Ag/AgCl) or rhodium (Rh). Layered electrodes, e.g., silver/silver chloride over gold, may also be used. In one example, each trans electrode 20 is selected from the group consisting of Ag and AgCl.

In the example shown in FIG. 1A, the support structure 12 supports a second substrate 34. In this example, the second substrate 34 functions as a lid and has the cis well 22 defined therein. The second substrate 34 also has the inlet 36 and outlet 38 defined therein so that fluid may be introduced into and extracted from the cis well 22. The inlet 36 and outlet 38 may be positioned at opposed ends of the cis well 22 or anywhere along the length and width of the cis well 22 that enables desirable fluid flow. While not shown, it is to be understood that the inlet 36 and outlet 38 are fluidly connected to a fluidic control system (including, e.g., reservoirs, pumps, valves, waste containers, and the like) which controls fluid introduction and expulsion.

The second substrate 34 may be any of the substrate materials set forth herein, such as polymer, glass, ceramic, or metal. The second substrate 34 (and its features, such as the cis well 22, the inlet 36, and the outlet 38) may be fabricated using injection molding or CNC (computer numerical control) machining. It is to be understood that the second substrate 34 may be fabricated monolithically using, for example, additive manufacturing techniques. Alternatively, the second substrate 34 may be an assembly of parts joined together using appropriate joining technology, including welding and/or adhesives. For example, a sub-assembly defining the top portion 34, T may be bonded, welded, or mechanically attached to an interposer (which defines the sidewalls 34, S).

The second substrate 34 is a separate piece from the patterned layer 28, but the two components 28, 34 may be formed of the same material or of different materials. The second substrate 34 may be prefabricated and then adhered to the patterned layer 28, or may be deposited and patterned using similar techniques to those described for the patterned layer 28. Examples of suitable adhesives include thermoset or thermoplastic polymers, tape, and a photocurable glue (e.g., acrylic based). Alternatively, the second substrate 34 and the patterned layer 28 may be laminated together, e.g., by applying heat and/or mechanical pressure. In the example shown in FIG. 1A, the substrate 34 is attached to the patterned layer 28 such that each of the trans wells 18 is in fluid communication with the cis well 22. While a single array of trans wells 18 is shown in fluid communication with the one cis well 22, it is to be understood that the fluidic device 10A may include several fluidically isolated cis wells 22, each of which is in fluid communication with a separate array of trans wells 18. Multiple cis wells 22 may be desirable, for example, in order to enable the measurement of multiple samples using a single nanopore fluidic device 10A.

When included, the cis well 22 may have any suitable geometry and/or dimensions. In the examples shown in FIG. 1A, the cis well 22 has a rectangular shaped geometry. The dimensions of the cis well 22 may range from about 1 mm×0.1 mm×0.1 mm to about 100 mm×10 mm×10 mm.

The fluidic device 10A also includes the cis electrode 24. The cis electrode 24 may be any of the examples set forth herein for the trans electrode 20.

In the example shown in FIG. 1A, the cis electrode 24 is attached to an interior surface of the top portion 34, T so that it forms a top of the cis well 22.

The nanopore fluidic device 10A shown in FIG. 1A may also include circuitry suitable for the operational use of the device 10A. Generally, the circuitry is in operable communication with the cis and trans electrodes 22, 20 and is configured to detect changes in an electrical characteristic of the opening 48 (see FIG. 2) of the nanopore 44. Such changes may, for example, be responsive to any suitable stimulus. Indeed, it will be appreciated that the present methods, compositions, and devices may be used in any suitable application or context, including any suitable method or device for polynucleotide sequencing.

The circuitry may be integrated into a semiconductor wafer that functions as or is in contact with the substrate 26. The circuitry includes at least a stimulus source (e.g., a voltage source 46) and a controller (not shown). The stimulus source may be coupled to the cis electrode 24 and to each trans electrode 20 either individually or via multiplexing, and the stimulus source is to cause current to flow through one or more of the nanopores 44 by addressing the trans electrode(s) 20 associated with a respective nanopore 44. The controller is coupled to the stimulus source, and the controller is configured to individually/selectively address one of the trans electrode(s) 20 (using the stimulus source) to cause an ionic current to flow through the nanopore 44 connected to the addressed trans electrode 20. In one example, each electrode 20 is electrically connected to its own set of electronics, which includes the stimulus source and the controller. In another example, each of the trans electrodes 20 is electrically connected to a single stimulus source and controller, which are connected to a multiplexer. The circuitry may also include operational amplifier(s) 47 to amplify electrical signals passing through respective nanopores 44 associated with electrodes 20 that are addressed.

In one example, the nanopore fluidic device 10A depicted in FIG. 1A may be available as shown, and a user may add the membranes 42 and nanopores 44 prior to use. In another example, the nanopore fluidic device 10A may have the membranes 42 in an operable position, and a user may add the nanopores 44 prior to use. In still another example, the nanopore fluidic device 10A may have the membranes 42 in an inoperable position, and a user may adjust the membranes 42 to an operable position and then add the nanopores 44 prior to use. In yet another example, the nanopore fluidic device 10A may have the membranes 42 and nanopores 44 in an inoperable position, and a user may adjust the membranes 42 and the nanopores 44 to an operable position prior to use. In yet a further example, the nanopore fluidic device 10A may have the membranes 42 and nanopores 44 in an operable position. The membranes 42 and nanopores 44 will be described in reference to FIG. 1B.

FIG. 1B depicts another example of the nanopore fluidic device 10B after the membranes 42 and nanopores 44 are in the operable position.

In the example shown in FIG. 1B, the support structure 12′ includes the substrate 26, a top layer 32 having the top hydrophobic surface 14 and a sidewall layer 30 having the hydrophilic sidewalls 16.

The substrate 26 may be any of the examples set forth herein in the definitions. While not shown in FIG. 1B, it is to be understood that any suitable securing mechanism may be used to adhere the substrate 26 and the sidewall layer 30 together.

The sidewall layer 30 may be any negative photoresist that inherently has a higher surface energy than the top layer 32 or that can be treated to increase the surface energy. Any of the negative photoresists disclosed herein may be used for the sidewall layer 30. In one example, SINR is used. Other negative photoresists can be rendered hydrophilic using a non-fluorinated plasma treatment.

The top layer 32 may be any negative photoresist that inherently has a lower surface energy than the sidewall layer 30 or that can be treated to decrease the surface energy. In one example, the top layer comprises a low surface energy material. The low surface energy material may be a fluorine-containing material. Examples of the top layer 32 include SU-8 3000, or fluoropolymers (e.g., CYTOP® available from AGC Chemicals Co., the FLUOROLINK™ Series from Solvay, polytetrafluoroethylene), or SX AR-PC 5060 F-Protect (an amorphous perfluorinated copolymer dissolved in a solvent mixture of perfluorotri-n-butylamine and perfluoro-n-dibutylmethylamine from Allresist GmbH).

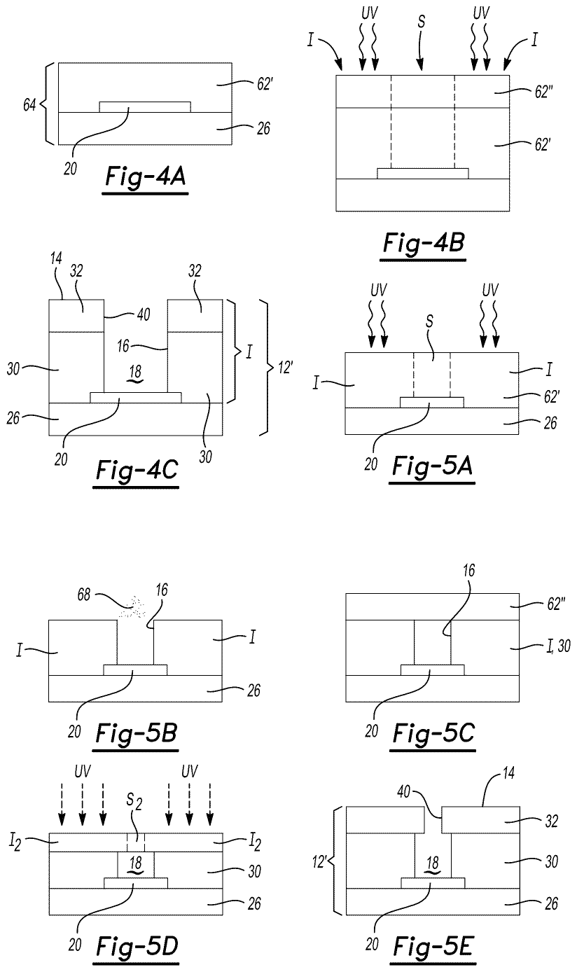

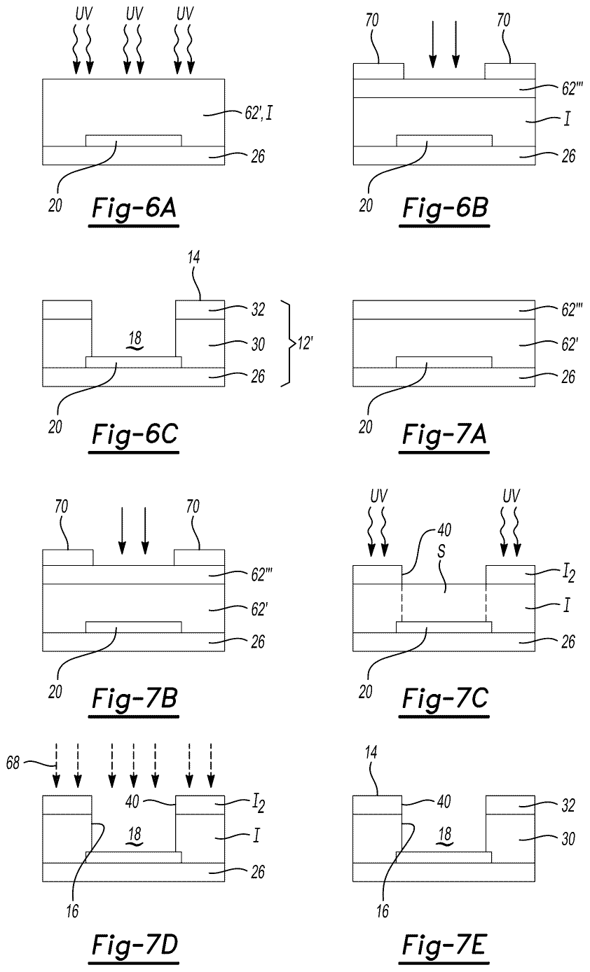

The top layer 32, which includes the top hydrophobic surface 14, and the sidewall layer 30 may be formed as described in any of the methods described in reference to the FIG. 4 series, the FIG. 5 series, the FIG. 6 series, or the FIG. 7 series.

The material selected for the sidewall layer 30 is also capable of being processed with nanometer or micrometer accuracy for defining the trans wells 18. The dimensions, aspect ratio, layouts, and aperture 40 of each of the trans wells 18 shown in FIG. 1B may be any of the examples described herein.

In the example shown in FIG. 1B, each trans well 18 includes the hydrophilic sidewalls 16, which, in which example, are defined by the sidewall layer 30.

As depicted in FIG. 1B, the lower surface of each trans well 18 is defined by the trans electrode 20. The trans electrodes 20 extend beyond the hydrophilic sidewalls 16 such that the sidewall layer 30 is positioned over the perimeter of each trans electrode 20 (see, e.g., FIG. 1B). However, the trans electrodes 20 are physically isolated from each other. The trans electrodes 18 may be any suitable electrode material set forth herein.

In the example shown in FIG. 1B, the support structure 12′ supports the second substrate 34. As described in reference to FIG. 1A, the second substrate 34 functions as a lid, and has the cis well 22, inlet 36, and outlet 38 defined therein.

In the fluidic device 10B, the second substrate 34 is a separate piece from the top layer 32, but the two components 32, 34 may be formed of the same material or of different materials. Any of the securing mechanisms described herein may also be used to adhere the second substrate 34 and the top layer 32 together. In the example shown in FIG. 1B, the substrate 34 is attached to the top layer 32 such that each of the trans wells 18 is in fluid communication with the cis well 22. While a single array of trans wells 18 is shown in fluid communication with the one cis well 22, it is to be understood that the fluidic device 10B may include several fluidically isolated cis wells 22, each of which is in fluid communication with a separate array of trans wells 18. Multiple cis wells 22 may be desirable, for example, in order to enable the measurement of multiple samples using a single nanopore fluidic device 10B.

The cis well 22 shown in FIG. 1B may have suitable geometry and/or dimensions described herein.

The fluidic device 10B also includes the cis electrode 24. The cis electrode 24 may be any of the examples set forth herein and may be secured to the top portion 34, T as described herein.

The circuitry described in reference to FIG. 1A may also be used in the fluidic device 10B.

The nanopore fluidic device 10B also includes the membranes 42 and the nanopores 44 in an operable position. More specifically, respective membranes 42 are formed over at least some of the plurality of trans wells 18, where each of the respective membranes 42 is supported by the top hydrophobic surface 14. Moreover, respective nanopores 44 are positioned in at least some of the respective membranes 42.

The membranes 42 shown in FIG. 1B are in the operable position across the aperture 40. When in the operable position across the aperture 40, each membrane 42 has a first (trans) side 50 facing the trans well 18 and a second (cis) side 52 facing the cis well 22. The membrane 42 may have any suitable structure that normally inhibits passage of molecules from one side of the membrane 42 to the other side of the membrane 42, e.g., that normally inhibits contact between fluids 54, 56 contained in the respective wells 18, 22. For example, as illustrated in FIG. 2, the structure of the membrane 42 may be a bilayer structure including a first layer 58 and a second layer 60, one or both of which inhibit(s) the flow of molecules across the respective layer. The first and second layers 58, 60 may be formed using examples of the AB diblock copolymers, or certain ABA triblock copolymers. Alternatively, the structure of the membrane 42 may include a single layered structure, which inhibits the flow of molecules across that layer. The single layered membrane 42 may be formed using certain ABA triblock copolymers. In still other examples, the structure of the membrane 42 may be partially a single layer, and partially a bilayer, formed using certain ABA triblock copolymers.

When the membrane 42 has a bilayer structure, the first layer 58 includes a first plurality of molecules of a diblock or triblock copolymer, and the second layer 60 includes a second plurality of molecules of the copolymer. In examples in which the copolymer is a diblock copolymer (which may be referred to as AB), each molecule may include a hydrophobic block coupled to a hydrophilic block. The hydrophilic blocks of the first plurality of molecules may form a first outer surface of membrane 42, e.g., the surface of layer 48 forming the first side 50. The hydrophilic blocks of the second plurality of molecules may form a second outer surface of membrane 42, e.g., the surface of layer 60 forming the second side 52. The hydrophobic blocks of the first and second pluralities of molecules may contact one another within the membrane 42. In examples in which the copolymer is a triblock copolymer (which may be referred to as ABA), each molecule may include first and second hydrophilic blocks and a hydrophobic block disposed therebetween. In the bilayer structure, each layer 58, 60 is composed of folded ABA molecules. More particularly, the B blocks of each of the first plurality of molecules are folded such that both A blocks of each molecule form a first outer surface of membrane 42, e.g., the surface of layer 58 forming the first side 50. Similarly, the B blocks of each of the second plurality of molecules are folded such that both A blocks of each molecule form a second outer surface of membrane 42, e.g., the surface of layer 60 forming the second side 52. The folded hydrophobic B blocks of the first and second plurality of molecules may contact one another within the membrane 42.

When the membrane 20 has a single layered structure, the single layer includes a plurality of molecules of the triblock copolymer. In this example, the ABA molecules extend from the trans well 18 to the cis well 22, forming surfaces 50, 52.

The diblock and triblock copolymers used to form examples of the membrane 42 may include any suitable combination of hydrophobic and hydrophilic blocks. In some examples, the hydrophilic A block may include a polymer selected from the group consisting of: N-vinyl pyrrolidone, polyacrylamide, zwitterionic polymer, hydrophilic polypeptide, nitrogen containing units, and poly(ethylene oxide) (PEO). Illustratively, the polyacrylamide may be selected from the group consisting of: poly(N-isopropyl acrylamide) (PNIPAM), and charged polyacrylamide, and phosphoric acid functionalized polyacrylamide. An example of a charged polyacrylamide is

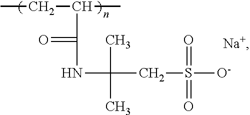

where n is between about 2 and about 100.

In some examples, the hydrophobic B block may include a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB). Examples of hydrogenated polydienes include saturated polybutadiene (PBu), saturated polyisoprene (PI), or saturated poly(myrcene).

In one example, an AB diblock copolymer includes PDMS-b-PEO, where “-b-” denotes that the polymer is a block copolymer. In another example, an AB diblock copolymer includes PBd-b-PEO. In still another example, an AB diblock copolymer includes PIB-b-PEO.

In one example, an ABA triblock copolymer includes PEO-b-PBd-b-PEO. In another example, an ABA triblock copolymer includes PEO-b-PDMS-b-PEO. In still another example, an ABA triblock copolymer includes PEO-b-PIB-b-PEO.

When in the operative position (as shown in FIG. 1B and FIG. 2), the nanopore 44 is disposed within the membrane 42 and provides an opening 48, which fluidically couples the respective trans well 18 with the cis well 22. As such, the opening 48 of the nanopore 42 may provide a pathway for fluid 56 and/or fluid 54 to flow through the membrane 42. For example, a portion of salt may move from the second side 52 of the membrane 42 to the first side 50 of the membrane 42 through the opening 48.

In the examples set forth herein, the nanopore(s) 42 is/are biological nanopore(s) or hybrid nanopore(s). The nanopore 42 may be any of the examples set forth herein in the definitions. An example of a biological nanopore is Mycobacterium smegmatis porin A (MspA).

When in operational use, the nanopore fluidic devices 10A, 10B include a first fluid 54 in the trans well 18 and a second fluid 56 in the cis well 22. The first fluid 54 within the trans well 18 is in contact with the first side 50 of the membrane 42, and the second fluid 56 within the cis well 22 is in contact with the second side 52 of the membrane 42.

The first fluid 54 may have a first composition including a first concentration of a salt. The second fluid 56 may have a second composition including a second concentration of the salt that may be the same as, or different than, the first concentration. Any suitable salt or salts may be used in the first and second fluids 54, 56, e.g., ranging from common salts to ionic crystals, metal complexes, ionic liquids, or even water soluble organic ions. For example, the salt may include any suitable combination of cations (such as H, Li, Na, K, NH₄, Ag, Ca, Ba, and/or Mg) with any suitable combination of anions (such as OH, Cl, Br, I, NO₃, ClO₄, F, SO₄, and/or CO₃²⁻ . . . ). In one example, the salt includes potassium chloride (KCl). It will also be appreciated that the first and second fluids optionally may include any suitable combination of other solutes. Illustratively, the first and second fluids 54, 56 may include an aqueous buffer (such as N-(2-hydroxyethyl) piperazine-N′-2-ethanesulfonic acid (HEPES), commercially available from Thermo Fisher Scientific Inc.). Any difference between the first and second concentrations of salt and/or between other components of the first and second fluids 54, 56, may generate osmotic pressure across the membrane 42.

While the nanopore fluidic device 10B depicted in FIG. 1B is shown with the membranes 42 and nanopores 44 in the operative position, it is to be understood that the nanopore fluidic device 10B may be available in any of the configurations described in reference to the nanopore fluidic device 10A.

### Methods for Making Nanopore Fluidic Devices

An example method for making the support structure 12 of the nanopore fluidic device 10A of FIG. 1A is described in reference to the FIG. 3 series.

The method shown in FIG. 3A and FIG. 3B generally includes depositing a photoresist 62 over a substrate 26; soft baking the photoresist 62; exposing a portion of the photoresist 62 to ultraviolet light UV, thereby forming an insoluble support structure I, whereby another portion S of the photoresist 62 remains soluble; removing the soluble other portion S of the photoresist 62, thereby forming a plurality of trans wells 18 in the insoluble support structure I; and using reactive ion etching at a zero watt (zero W) substrate bias value to expose a top surface of the insoluble support structure I to a directional fluorine-based plasma, thereby decreasing a surface energy of the top surface of insoluble support structure I without decreasing the surface energy of sidewalls 16 of the insoluble support structure I that define the plurality of trans wells 18. After reactive ion etching, the patterned layer 28 with the hydrophobic top surface 14 and the hydrophilic sidewalls 16 is formed.

The photoresist 62 may be any of the negative photoresists set forth herein for the patterned layer 28. The photoresist 62 may be dissolved in a suitable solvent, such as cyclopentene, propylene glycol monomethyl ether acetate (PGMEA), or γ-butyrolactone (GBL). The photoresist 62 may be deposited using a suitable technique, such as chemical vapor deposition, dip coating, dunk coating, spin coating, spray coating, puddle dispensing, ultrasonic spray coating, doctor blade coating, aerosol printing, screen printing, microcontact printing, etc. To develop the negative photoresist 62 and pattern the trans wells 18, an ultraviolet (UV) light dosage (shown as UV in FIG. 3A) is directed to portions of the photoresist 62 that are to become insoluble. The portions S of the photoresist 62 that are not exposed to the UV light dosage remain soluble.

After UV light exposure and prior to removing the soluble portions S, the photoresist 62 (including insoluble I and soluble S portions) may be exposed to a post-exposure bake. This process may be performed to improve the adhesion between the substrate 26 and the insoluble portions I, and to diffuse the photo acid generated during the UV light dosage to fully crosslink the polymer chains of the insoluble portion I. The post-exposure bake may be performed at a temperature ranging from about 65° C. to about 150° C. for a time ranging from about 1 minute to about 15 minutes.

The soluble portions S are then removed, e.g., with the developer. Examples of suitable developers for the negative photoresist include solvent based developers, such as PGMEA, ethyl lactate, and diacetone alcohol, aqueous-alkaline solutions, such as diluted sodium hydroxide, diluted potassium hydroxide, or an aqueous solution of the metal ion free organic TMAH (tetramethylammoniumhydroxide). After developer exposure, the insoluble portions I remain over the substrate 26. As shown in FIG. 3B, the removal of the soluble portions S forms the trans wells 18 and exposes the hydrophilic sidewalls 16.

After the soluble portions S are removed, the remaining structure may be hard baked. The duration of the hard bake may last from about 5 seconds to about 200 minutes at a temperature ranging from about 90° C. to about 300° C. An example of processing equipment that can be used for hard baking includes an oven.

The method then includes reactive ion etching at a zero W (0 W) substrate bias value to expose a top surface of the insoluble support structure 62, I to a directional fluorine-based plasma 66. The bias power of the reactive ion etching process can be tuned slightly, as long as it enables directional processing. The bias voltage energizes ions from the plasma 66, which are then accelerated towards the negatively biased top surface. The ions collide with the top surface, reacting chemically to reduce the surface energy and form the top hydrophobic surface 14. The ions are directed perpendicularly to the plane of the top surface, and thus have little to no effect on the hydrophilic sidewalls 16 (which are parallel to the direction of the ions that are introduced). As such, the directional fluorine-based plasma 66 can decrease the surface energy at the top surface of the insoluble support structure 62, I without decreasing the surface energy of the sidewalls 16. Examples of the fluorine-based plasma include sulfur hexafluoride (SF₆), tetrafluoromethane (CF₄), or nitrogen trifluoride (NF₃). Example plasma conditions include: ICP power: 250 W, RIE power: 0 W, pressure: 50 m Torr, and flow rate: 25 sccm.

It is believed that the method of FIG. 3A and FIG. 3B may be performed with a non-fluorine-based plasma, such as tetramethyl silane (Si(CH₃)₄).

The method shown in FIG. 3A and FIG. 3B is performed with the electrode 20 positioned on the substrate 26 before the photoresist 62 is applied thereon. In this example, a plurality of isolated electrodes 20 is positioned on the substrate 26 (one of which is shown in FIG. 3A and FIG. 3B); the photoresist 62 is deposited over each of the plurality of isolated electrodes 20 and the substrate 26; and each of the plurality of isolated electrodes 20 is exposed when each of the plurality of trans wells 18 is formed. In this example, at least a portion of the photoresist 62 that overlies each electrode 20 is not exposed to the UV light, and portions of the photoresist 62 that overly the substrate 26, which separates adjacent electrodes 20, are exposed to the UV light. This exposure pattern creates soluble photoresist 62, S over each of the electrodes 20. The removal of the soluble portions 62, S exposes the trans wells 18 and at least a portion of the surface of each of the trans electrodes 20. An example structure is shown in FIG. 3C, where the perimeter of the trans electrode 20 is covered by the patterned layer 28, but the remainder of the trans electrode 20 is exposed in the trans well 18.

Example methods for making the support structure 12′ of the nanopore fluidic device 10B of FIG. 1B is described in reference to the FIG. 4 series, the FIG. 5 series, the FIG. 6 series, and the FIG. 7 series.

The method described in reference to FIG. 4A through FIG. 4C generally includes forming a material stack 64 by depositing a first photoresist 62′ having a first surface energy over a substrate 26; soft baking the first photoresist 62′; depositing a second photoresist 62″ having a second surface energy that is lower than the first surface energy over the first photoresist 62′; soft baking the second photoresist 62″; exposing a portion of the material stack 64 to ultraviolet light UV, thereby forming an insoluble support structure I, whereby another portion S of the material stack 64 remains soluble; and removing the soluble other portion S, thereby forming a plurality of trans wells 18 in the insoluble support structure I.

In this example method, two different photoresists 62′, 62″ are used. The photoresists 62′, 62″ may be any of the examples set forth herein, as long as the photoresist 62′ has a higher surface energy than the photoresist 62″. As an example, the photoresist 62′ may be any of the examples set forth herein for the patterned layer 28 and the sidewall layer 30, and the photoresist 62″ may be any of the examples set forth herein for the top layer 32.

The photoresist 62′ may be deposited over the substrate using a suitable technique described herein. The photoresist 62′ is exposed to a soft bake in order to stabilize the resist for deposition of the photoresist 62″. The soft bake may take place at a temperature ranging from about 50° C. to about 150° C., for greater than 0 seconds to about 60 minutes. Examples of devices that can be used for soft baking include a hot plate, an oven, etc.

The photoresist 62″ is then deposited over the soft baked photoresist 62′. Any suitable technique described herein may be used to deposit the photoresist 62″. The photoresist 62″ is exposed to a soft bake. This soft bake may be performed as described herein.

To develop the negative photoresists 62′, 62″ and pattern the trans wells 18, an ultraviolet light dosage (shown as UV in FIG. 4B) is directed to portions of the photoresists 62′, 62″ that are to become insoluble. The portions S of the photoresist 62′, 62″ that are not exposed to the UV light dosage remain soluble.

After UV light exposure and prior to removing the soluble portions S, the photoresists 62′, 62″ (including insoluble I and soluble S portions) may be exposed to a post-exposure bake. This process may be performed to improve the adhesion between the substrate 26 and the insoluble portions I.

The soluble portions S are then removed, e.g., with an example of the developer set forth herein. Depending upon the materials used, a single developer may be used to remove the soluble portions S of both photoresists 62″, 62′; or two different developers may be used to remove the soluble portions of the respective photoresists 62″, 62′.

After developer exposure, the insoluble portions I of both photoresists 62′, 62″ remain over the substrate 26. After the soluble portions S are removed, the remaining structure may be hard baked. The duration of the hard bake may last from about 5 seconds to about 200 minutes at a temperature ranging from about 90° C. to about 300° C. Examples of devices that can be used for hard baking include a hot plate, an oven, etc.

As shown in FIG. 4C, the removal of the soluble portions S forms the trans wells 18 and exposes the hydrophilic sidewalls 16. Because the photoresists 62′, 62′ are selected to form, respectively, the hydrophilic sidewalls 16 and the hydrophobic top surface 14, additional processing is not performed after hard baking. As such, the method of FIG. 4A through FIG. 4C generates the support structure 12′, including the sidewall layer 30 (from the photoresist 62′) and the top layer 30 (from the photoresist 62″).

As depicted, the method shown in FIG. 4A through FIG. 4C is performed with the trans electrode 20 positioned on the substrate 26 before the photoresists 62′, 62″ are applied thereon. In this method, the trans electrode 20 is positioned on the substrate 26, and the first photoresist 62′ is deposited over the trans electrode 20 and the substrate 26. In a more specific example, a plurality of isolated electrodes 20 is positioned on the substrate 26; the photoresist 62′ is deposited over each of the plurality of isolated electrodes 20 and the substrate 26; and each of the plurality of isolated electrodes 20 is exposed when each of the plurality of trans wells 18 is formed. In this example, portions of the photoresists 62′, 62″ that overlie each electrode 20 are not exposed to the UV light, and portions of the photoresists 62′, 62″ that overlie the substrate 26, which separates adjacent electrodes 20, are exposed to the UV light. This exposure pattern creates soluble photoresist S over at least a portion of each of the electrodes 20. The removal of the soluble portions S exposes the trans wells 18 and at least a portion of the surface of each of the trans electrodes 20.

Referring now to the FIG. 5 series, the method described in reference to FIG. 5A through FIG. 5E generally includes depositing a first photoresist 62′ over a substrate 26; soft baking the first photoresist 62′; exposing a portion of the first photoresist 62′ to ultraviolet light UV, thereby forming an insoluble support structure I, whereby another portion S of the first photoresist 62′ remains soluble; removing the soluble other portion S of the first photoresist 62′, thereby forming a plurality of trans wells 18 in the insoluble support structure I; exposing a surface of the insoluble support structure I to a plasma 68, thereby increasing a surface energy of the insoluble support structure I; and laminating a second photoresist 62″ to the surface of the insoluble support structure I.

Similar to the FIG. 4 series, the method of the FIG. 5 series uses two different photoresists 62′, 62″. The photoresists 62′, 62″ may be any of the examples set forth herein, as long as the photoresist 62′ has a higher surface energy after plasma exposure than the photoresist 62″. As an example, the photoresist 62′ may be any of the examples set forth herein for the patterned layer 28 and the sidewall layer 30, and the photoresist 62″ may be any of the examples set forth herein for the top layer 32.

The photoresist 62′ may be deposited over the substrate 26 using a suitable technique described herein. The photoresist 62′ is exposed to a soft bake as described herein.

In this example method, the photoresist 62′ is then patterned with ultraviolet light UV. To develop the photoresists 62′ and pattern a portion of the trans wells 18, an ultraviolet light dosage (shown as UV in FIG. 5A) is directed to portions of the photoresist 62′ that are to become insoluble. The portions S of the photoresist 62′ that are not exposed to the UV light dosage remain soluble.

After UV light exposure and prior to removing the soluble portions S, the photoresists 62′ (including insoluble I and soluble S portions) may be exposed to a post-exposure bake. This process may be performed to improve the adhesion between the substrate 26 and the insoluble portions I and to diffuse the photo acid generated during the UV light dosage to fully crosslink the polymer chains of insoluble portion I.

The soluble portions S are then removed, e.g., with an example of the developer set forth herein.

After developer exposure, the remaining insoluble portions I are exposed to a plasma 68 that will increase the surface energy of the insoluble portions I, and thus create the hydrophilic sidewalls 16. The plasma 68 may be an argon plasma or an oxygen plasma. In addition to increasing the surface energy of the insoluble portions I, the plasma 68 may clean trans electrodes 20 that are positioned on the substrate 26 at the outset of the method. If the trans electrode(s) 20 are formed of oxidative sensitive materials, the argon plasma is selected. In this example, the plasma 68 should be directed at least within the trans well 18 to increase the surface energy of the sidewalls 16. The top surface of the insoluble portions I may or may not be exposed to the plasma 68. The plasma exposed insoluble portions I form the sidewall layer 30.

The photoresist 62″ is then laminated to the top surface of the sidewall layer 30. In this example, the photoresist 62″ includes minimal amounts of solvent, and is covered with plastic liners. A first liner may be removed prior to lamination so that the photoresist 62″ and top surface can be placed into direct contact, and the a second liner may be removed post-lamination. Example lamination conditions include: temperature ranging from about 30° C. to about 130° C., speed ranging from about 0.5 cm/s to about 10 cm/s, and pressure ranging from about 5 psi to about 75 psi.

The structure shown in FIG. 5C (with the photoresist 62″ positioned over the sidewall layer 30 and the trans well(s) 18) is exposed to soft baking as described herein.

This method then includes exposing a portion of the second photoresist 62″ to ultraviolet light UV, thereby forming an insoluble layer (portions I₂) on the surface of the insoluble support structure I (formed from the first photoresist 62′), whereby another portion S₂ of the second photoresist 62″ remains soluble; and removing the soluble other portion S₂ of the second photoresist 62″, thereby forming an aperture 40 of each of the plurality of trans wells 18. The UV light exposure of the photoresist 62″ patterns the insoluble portions I₂, which will form the top layer 32 and the top hydrophobic surface 14. Those areas not exposed to the UV light will pattern the soluble portions S₂, which will form the aperture 40 and the remainder of the trans well 18. UV light exposure is depicted in FIG. 5D. In the example shown in FIG. 5D, the diameter of the soluble portion S₂ is smaller than the diameter of the trans well 18, and thus the top layer 32 will form an overhang that extends over a portion of the trans well 18. The overhang creates a large trans well 18 with a narrow aperture 40. Alternatively, the diameter of the soluble portion S₂ can be the same as the diameter of the trans well 18. This will create a trans well 18 whose aperture 40 is the same size as the rest of the well 18.

After UV light exposure and prior to removing the soluble portions S, the photoresists 62′, 62″ (including insoluble I₂ and soluble S₂ portions) may be exposed to a post-exposure bake.

The soluble portions S are then removed, e.g., with an example of the developer set forth herein. The removal of the soluble portions S creates the aperture 40 and the remainder of the trans well 18, as shown in FIG. 5E.

After the soluble portions S are removed, the remaining structure may be hard baked as described herein.

Because the photoresist 62′ is selected to form the top hydrophobic surface 14, additional processing is not performed after hard baking. As such, the method of FIG. 5A through FIG. 5E generates the support structure 12′, including the sidewall layer 30 and hydrophilic sidewalls 16 (from the photoresist 62′), and the top layer 30 and top hydrophobic surface 14 (from the photoresist 62″).

As depicted in FIG. 5A through FIG. 5E, the method is performed with the electrode 20 positioned on the substrate 26 before the photoresists 62′, 62″ are applied thereon. In this method, the trans electrode 20 is positioned on the substrate 26, and the first photoresist 62′ is deposited over the trans electrode 20 and the substrate 26. In a more specific example, a plurality of isolated trans electrodes 20 is positioned on the substrate 26; the photoresist 62′ is deposited over each of the plurality of isolated trans electrodes 20 and the substrate 26; and each of the plurality of isolated trans electrodes 20 is exposed when each of the plurality of trans wells 18 is formed. In this example, portions of the photoresists 62′, 62″ that overlie each trans electrode 20 are not exposed to the UV light during the respective exposure events, and portions of the photoresists 62′, 62″ that overlie the substrate 26, which separates adjacent trans electrodes 20, are exposed to the UV light during the respective exposure events. These exposure patterns create soluble photoresist S, S₂ over at least a portion of each of the electrodes 20. The removal of the soluble portions S, S₂ exposes the trans wells 18 and at least a portion of the surface of each of the trans electrodes 20.

Referring now to the FIG. 6 series, the method described in reference to FIG. 6A through FIG. 6C generally includes depositing a first photoresist 62′ over a substrate 26; soft baking the first photoresist 62′; exposing the first photoresist 62′ to ultraviolet light UV, thereby forming an insoluble first photoresist I; baking the insoluble first photoresist I; applying a fluoropolymer photoresist 62′″ on the insoluble first photoresist I; soft baking the fluoropolymer photoresist 62′″; using an etch mask 70 and an oxygen plasma, selective etching through each of the fluoropolymer photoresist 62′″ and the insoluble first photoresist I to define a plurality of apertures 40 in the fluoropolymer photoresist respectively leading to a plurality of trans wells 18 in the insoluble first photoresist I; removing the etch mask 70; and baking the insoluble first photoresist I and the fluoropolymer photoresist 62′″, thereby lowering a surface energy of the fluoropolymer photoresist 62′″.

The photoresist 62′ may be deposited over the substrate 26 using a suitable technique described herein. The photoresist 62′ is exposed to a soft bake as described herein. In this example method, the entire photoresist 62′ is exposed to UV light so that the entire photoresist 62′ becomes insoluble, forming the insoluble first photoresist I. The insoluble first photoresist I may be exposed to a soft bake as described herein.

The fluoropolymer photoresist 62′″ is then applied to the insoluble first photoresist I. The fluoropolymer photoresist 62′″ can be laminated on the insoluble first photoresist I, or incorporated into a solvent and deposited using any example deposition technique described herein. The fluoropolymer photoresist 62′″ may be exposed to a soft bake as described herein.

An etch mask 70 is then applied to the outer surface of the fluoropolymer photoresist 62′″. The pattern of the etch mask 70 covers portions of the fluoropolymer photoresist 62′″ that are not to be etched away. Portions of the fluoropolymer photoresist 62′″ and the underlying insoluble first photoresist I that are not covered by the etch mask 70 are able to be etched. The etch mask 70 could be a(n) photoresist, metal, or oxide. The etch mask 70 can be applied by a photolithography process.

The method then involves etching the exposed portion of the fluoropolymer photoresist 62′″, and then continuing etching through the portion of the insoluble first photoresist I that had been underneath the exposed portion of the fluoropolymer photoresist 62′″. In this example, the photoresists 62′″ and I may be etched using a dry, oxygen-based etching process, such as an anisotropic oxygen plasma, and the underlying trans electrode 20 acts as an etch stop.

Etching forms the trans well 18 and the aperture 40 of the trans well 18, and exposes the trans electrode 20 present at the outset of the method. The etch mask 70 may then be removed. Metals and oxide hard masks may be removed using a selective etching process. A photoresist mask may be removed using a solvent based strip.

After the etch mask 70 is removed, the structure is exposed to an annealing process. The annealing process may be performed at a temperature ranging from about 150° C. to about 250° C. for a time ranging from about 30 minutes to about 2 hours. The oxygen plasma used for etching can increase the surface energy of both photoresists 62′″, I, which is desirable for the hydrophilic sidewalls 16, but not the top hydrophobic surface 14. When exposed to annealing, the surface energy of the fluoropolymer photoresist 62′″ can be lowered, thus returning the material to its initial state. This annealing process creates the top layer 32 and the top hydrophobic surface 14. The annealing process hard bakes the insoluble first photoresist I, which creates the sidewall layer 30. The method shown in FIG. 6A through FIG. 6C forms the support structure 12′.

As depicted, the method shown in FIG. 6A through FIG. 6C is performed with the trans electrode 20 positioned on the substrate 26 before the photoresists 62′, 62′″ are applied thereon. In this method, the trans electrode 20 is positioned on the substrate 26, and the first photoresist 62′ is deposited over the trans electrode 20 and the substrate 26. In a more specific example, a plurality of isolated trans electrodes 20 is positioned on the substrate 26; the photoresist 62′ is deposited over each of the plurality of isolated trans electrodes 20 and the substrate 26; and each of the plurality of isolated trans electrodes 20 is exposed when each of the plurality of trans wells 18 is formed. In this example, portions of the photoresists 62′, 62′″ that overlie each trans electrode 20 are etched away.

Referring now to the FIG. 7 series, the method described in reference to FIG. 7A through FIG. 7E generally includes depositing a first photoresist 62′ over a substrate 26; soft baking the first photoresist 62′; applying a fluoropolymer photoresist 62′″ on the first photoresist 62′; soft baking the fluoropolymer photoresist 62′″; using an etch mask 70, selective etching through the fluoropolymer photoresist 62′″ to define a plurality of apertures 40 in the fluoropolymer photoresist 62′″; removing the etch mask 70; exposing a portion of the first photoresist 62′ to ultraviolet light UV, thereby forming an insoluble support structure I, whereby other portions S of the first photoresist 62′ that are adjacent to each of the plurality of apertures 40 remains soluble; removing the soluble other portions S of the first photoresist 62′, thereby forming a plurality of trans wells 18 in the insoluble support structure I that is respectively aligned with the plurality of apertures 40; exposing the plurality of trans wells 18 to a plasma, thereby increasing the surface energy of the insoluble support structure I; and baking the insoluble support structure I and the fluoropolymer photoresist 62′″, thereby lowering a surface energy of the fluoropolymer photoresist 62′″.

The photoresist 62′ may be deposited over the substrate 26 using any suitable technique described herein. The photoresist 62′ is exposed to a soft bake as described herein.

The fluoropolymer photoresist 62′″ is then applied to the photoresist 62′. The fluoropolymer photoresist 62′″ can be laminated on the photoresist 62′, or incorporated into a solvent and deposited using any example deposition technique described herein. The fluoropolymer photoresist 62′″ may be exposed to a soft bake as described herein. The material stack is shown in FIG. 7A.

An etch mask 70 is then applied to the outer surface of the fluoropolymer photoresist 62′″. The etch mask 70 may be any of the examples set forth herein. The pattern of the etch mask 70 covers portions of the fluoropolymer photoresist 62′″ that are not to be etched away. Portions of the fluoropolymer photoresist 62′″ that are not covered by the etch mask 70 are able to etched.

The method then involves etching the exposed portion of the fluoropolymer photoresist 62′″. In this example, the photoresists 62′″ may be etched using a dry, oxygen-based etching process, such as an anisotropic oxygen plasma. Etching may be performed for a time that is based on the etch rate and the thickness of the photoresist 62′″. Alternatively, an endpoint detector may be used in the etch chamber. The endpoint detector senses when the etched molecules have changed, thus indicating that etching is complete. When etching is stopped, the surface of the photoresist 62′ is exposed. This is depicted in FIG. 7B.

The etch mask 70 may then be removed. Metals and oxide hard masks may be removed using a selective etching process. A photoresist mask may be removed using a solvent based strip.

After the etch mask 70 is removed, the photoresists 62′, 62′″ are exposed to UV light to pattern insoluble regions I, I₂. The lack of UV exposure at the exposed portions of the photoresist 62′ generates soluble portions S.

After UV light exposure and prior to removing the soluble portions S, the photoresists 62′, 62′″ (including insoluble I, I₂ and soluble S portions) may be exposed to a post-exposure bake. The post-exposure bake may be performed at a temperature ranging from about 150° C. to about 250° C. for a time ranging from about 30 minutes to about 2 hours.

The soluble portions S are then removed, e.g., with the developer. This forms the trans well(s) 18.

As shown in FIG. 7D, the structure is then exposed to a plasma 68 that will increase the surface energy of the insoluble portions I, and thus create the hydrophilic sidewalls 16. The plasma 68 may be an argon plasma or an oxygen plasma. If the trans electrode 20 is formed of an oxidative sensitive material, the argon plasma is selected. In addition to increasing the surface energy of the insoluble portions I, the plasma 68 may clean trans electrodes 20 that are positioned on the substrate 26 at the outset of the method. In this example, the plasma 68 should be directed at least within the trans well 18 to increase the surface energy of the sidewalls 16. The top surface of the insoluble portions 12 may or may not be exposed to the plasma 68. The plasma exposed insoluble portions I form the sidewall layer 30 (shown in FIG. 7E).

The structure is exposed to an annealing process. The annealing process may be performed at a temperature ranging from about 150° C. to about 250° C. for a time ranging from about 30 minutes to about 2 hours. The plasma 68 can increase the surface energy of the insoluble portions I₂, which is undesirable for the top hydrophobic surface 14. When exposed to annealing, the surface energy of the fluoropolymer photoresist 62′″ can be lowered, thus returning the material to its initial state. This annealing process creates the top layer 32 and the top hydrophobic surface 14. The annealing process also hard bakes the insoluble first photoresist I. The method shown in FIG. 7A through FIG. 7E forms the support structure 12′.

As depicted, the method shown in FIG. 7A through FIG. 7E is performed with the trans electrode 20 positioned on the substrate 26 before the photoresists 62′, 62′″ are applied thereon. In this method, the trans electrode 20 is positioned on the substrate 26, and the first photoresist 62′ is deposited over the trans electrode 20 and the substrate 26. In a more specific example, a plurality of isolated trans electrodes 20 is positioned on the substrate 26; the photoresist 62′ is deposited over each of the plurality of isolated trans electrodes 20 and the substrate 26; and each of the plurality of isolated trans electrodes 20 is exposed when each of the plurality of trans wells 18 is formed. In this example, portions of the photoresists 62′, 62′″ that overlie each electrode 20 are either solubilized and removed or etched away.

While not shown in any of the FIG. 3 through FIG. 7 series, it is to be understood that the membranes 42 and nanopores 44 may be introduced before or after a second substrate 34 is attached to the top hydrophobic surface 14. Thus, each example method further includes forming a respective membrane 42 over a respective aperture 40 of at least some of the plurality of trans wells 18; and inserting a nanopore 44 in at least some of the respective membranes 42.

A membrane material may be mixed with a hydrophobic (non-polar) solvent, such as octane, to form a membrane mixture. The membrane material may be painted across the aperture 40. Suitable techniques for painting the membrane 40 include brush painting (manual), mechanical painting (e.g., using a stirring bar), bubble painting, or water-oil-water painting. With bubble painting, an aqueous solution is flowed through the channel (e.g., cis well 22, followed by an air (or other gas) gap, followed by the membrane mixture, and then another air (or other gas) gap. This process creates a bubble with the membrane mixture at its exterior. Additional aqueous solution is introduced into the channel to push the bubble and membrane mixture through the channel. As the bubble passes over the aperture 40, the membrane 42 is formed. With water-oil-water painting, an aqueous solution is first flowed through the channel (e.g., cis well 22), followed by the membrane mixture, followed by additional aqueous solution. The second aqueous solution pushes the membrane material across the aperture 40. These techniques result in the formation of the membrane 40, as shown in FIG. 1B and FIG. 2. The membrane 40 is supported by the support structure 12, 12′ and is suspended across the aperture 40. In some examples, the membrane 40 has a painting yield of 90% or more, or 95% or more. As such, some of the trans wells 18 may not have a membrane 40 positioned thereon.

An annulus 72 including the hydrophobic (non-polar) solvent, and which also may include other compound(s), may adhere to support structure 12, 12′ (e.g., at patterned layer 28 or top layer 32). The annulus 72 may also support a portion of the membrane 40, e.g., may be located within the membrane 40. This is depicted in FIG. 2, between layer 58 and layer 60).

The nanopore 42 is then inserted into the membrane 40. Suitable techniques for inserting a nanopore 42 into a suspended membrane 40 include electroporation, pipette pump cycle, and detergent assisted pore insertion. In one example, a solution of nanopores 42 in a buffer is added to the wells 18 or a channel overlying the wells 18, and the pores 42 are respectively inserted into individual membranes 40 using electrical pulses.

After initial nanopore 42 insertion, the excess of the nanopore solution is washed away with buffer.

Tools for forming suspended membranes 40 using synthetic polymers, and for inserting nanopores 42 in the suspended membranes 40 are commercially available, such as the Orbit 16 TC platform available from Nanion Technologies Inc. (California, USA).

The method also includes attaching a second substrate 34 to the insoluble support structure 12, 12′, thereby forming a cis well 22 in fluid communication with each of the plurality of trans wells 18. The attachment of the second substrate 34 may occur before or after membrane 40 formation and nanopore 42 insertion.

To secure the second substrate 34 to the support structure 12, 12′, an adhesive or laminate may be used. Feet portion 74 (see FIG. 1A and FIG. 1B) of the second substrate 34 are attached to portions of the patterned layer 18 or the top layer 32 at a perimeter of the device 10A, 10B or at a perimeter of each of several sub-sets of trans wells 18.

Prior to securing the components 34 and 12 or 12′ together, a cis electrode 24 may be formed on or attached to the interior of the substrate 34 so that it will be positioned within the cis well 22 in the final device 10A, 10B. The cis electrode 24 may be formed via metal deposition or may be pre-formed and secured to the substrate 34 using any suitable adhesive.

One type of nanopore sequencing is strand sequencing. One type of strand sequencing involves the use of a polynucleotide binding protein, such as a motor protein or a helicase to control the movement of the polynucleotide through the nanopore. A polynucleotide binding protein may be used to simultaneously separate the double stranded polynucleotide and control the rate of translocation of the resultant single strand through the nanopore. When a potential is applied across a nanopore, there is a change in the current flow when an analyte, such as a nucleotide, resides transiently in nanopore for a certain period of time. The single polynucleotide strand is passed through the pore and the identities of the nucleotides are derived. Nanopore sequencing instruments that employ strand sequencing includes the MinION™, GridION™, and PromethION™ from Oxford Nanopore Technologies (Oxford, United Kingdom) and the CycloneSEQ™ from BGI Group (Shenzhen, China) and its subsidiaries.

Another type of strand sequencing involves modifying nucleotides on a strand of polynucleotide to carry a reporter, where the reports include tags or labels to produce a detectable signal. The modified polynucleotide can be translocated through the nanopore (i.e., protein nanopore, solid state nanopore, hybrid nanopore) without a polynucleotide binding protein to control the movement of the polynucleotide. An example of modified nucleotides on a strand of polynucleotide are described in U.S. Patent Application Publication US 2009/0035777 A1 assigned to Roche Sequencing Solutions Inc.

Another type of strand sequencing involves modifying nucleotides on a strand of polynucleotide to carry a modification, where the modifications can arrest or slow translocation when encountering the nanopore. The modified polynucleotide can be translocated through the nanopore (i.e., protein nanopore, solid state nanopore, hybrid nanopore) without a polynucleotide binding protein to control the movement of the polynucleotide. In some versions, application of a voltage can move one nucleotide and its attached modification through the nanopore at a time. The modified polynucleotide nucleotides on a strand of polynucleotide are described in PCT Publication No. WO 2024/228928 by Illumina, Inc.

One type of nanopore sequencing involves disposing a polynucleotide within a nanopore. The polynucleotide includes a formed duplex. The duplex may be extended with a polymerase within the nanopore instrument. When a potential is applied across a nanopore, the duplex or extended duplex is held within the nanopore since the size of the constriction of the nanopore inhibits passage of the duplex completely through the nanopore. The electrical signal of the duplex held within the nanopore is used to identify the polynucleotide sequencing. Detecting a polynucleotide duplex is described in PCT Publication No. WO 2023/049682A1A1 by Illumina, Inc.

Some of the nanopore sequencing described herein utilize binding proteins (e.g., motor proteins, helicases) and polymerases. Other types of nanopore sequencing may use exonucleases.

Analytes have been described herein as polynucleotides. Analytes may further include peptides, polypeptides, proteins, and constructs thereof.

### CLAUSES

Certain aspects of the present subject matter are expressed in the following clauses. The present invention is not intended to be limited to such clauses, unless expressly recited in the claims.

Clause 1. A nanopore fluidic device, comprising:
a support structure having a top hydrophobic surface and having hydrophilic sidewalls defining a plurality of trans wells;
a trans electrode associated with each of the plurality of trans wells; one or more cis wells facing the plurality of trans wells; and
a cis electrode associated with each of the one or more cis wells.

Clause 2. The nanopore fluidic device as defined in clause 1, further comprising respective membranes formed over at least some of the plurality of trans wells, each of the respective membranes being supported by the top hydrophobic surface.

Clause 3. The nanopore fluidic device as defined in clause 2, further comprising respective nanopores positioned in at least some of the respective membranes.

Clause 4. The nanopore fluidic device as defined in one of clauses 1-3, wherein the top hydrophobic surface comprises a low surface energy plasma treatment.

Clause 5. The nanopore fluidic device as defined in clause 4, wherein:
the low surface energy plasma treatment comprises a fluorine plasma treatment; or
the low surface energy plasma treatment comprises a tetramethyl silane.

Clause 6. The nanopore fluidic device as defined in one of clauses 1-3, wherein the support structure comprises a top layer having the top hydrophobic surface and a sidewall layer having the hydrophilic sidewalls.

Clause 7. The nanopore fluidic device as defined in clause 6, wherein the top layer comprises a low surface energy material.

Clause 8. The nanopore fluidic device as defined in clause 7, wherein the low surface energy material comprises a fluorine-containing material.

Clause 9. The nanopore fluidic device as defined in clause 6, wherein the sidewall layer comprises a high surface energy material.

Clause 10. The nanopore fluidic device as defined in clause 6, wherein the sidewall layer comprises a first photoresist and the top layer comprises a second photoresist layer.

Clause 11. The nanopore fluidic device as defined in one of clauses 1-3, wherein the support structure comprises a photoresist.

Clause 12. The nanopore fluidic device as defined in one of clauses 1-11, wherein each trans electrode is selected from the group consisting of Ag and AgCl.

Clause 13. The nanopore fluidic device as defined in one of clauses 1-12, wherein the each of the plurality of trans wells has a diameter of 50 μm or less.

Clause 14. The nanopore fluidic device as defined in one of clauses 1-13, wherein an aspect ratio of each of the plurality of trans wells is 1:1 or more.

Clause 15. A method, comprising:
forming a material stack by:
   depositing a first photoresist having a first surface energy over a substrate;
   soft baking the first photoresist;
   depositing a second photoresist having a second surface energy that is lower than the first surface energy over the first photoresist; and
   soft baking the second photoresist;
exposing a portion of the material stack to ultraviolet lightultraviolet (UV) light, thereby forming an insoluble support structure, whereby another portion of the material stack remains soluble; and
removing the soluble other portion, thereby forming a plurality of trans wells in the insoluble support structure.

Clause 16. The method as defined in clause 15, wherein an electrode is positioned on the substrate, and wherein the first photoresist is deposited over the electrode and the substrate.

Clause 17. The method as defined in one of clauses 15-16, further comprising:
forming a respective membrane over a respective aperture of at least some of the plurality of trans wells; and
inserting a nanopore in at least some of the respective membranes.

Clause 18. The method as defined in one of clauses 15-17, further comprising attaching a second substrate to the insoluble support structure, thereby forming a cis well in fluid communication with each of the plurality of trans wells.

Clause 19. The method as defined in clause 18, further comprising securing a cis electrode in the second substrate prior to the attaching.

Clause 20. A method, comprising:
depositing a first photoresist over a substrate;
soft baking the first photoresist;
exposing a portion of the first photoresist to ultraviolet lightultraviolet (UV) light, thereby forming an insoluble support structure, whereby another portion of the first photoresist remains soluble;
removing the soluble other portion of the first photoresist, thereby forming a plurality of trans wells in the insoluble support structure;
exposing a surface of the insoluble support structure to a plasma, thereby increasing a surface energy of the insoluble support structure; and
laminating a second photoresist to the surface of the insoluble support structure.

Clause 21. The method as defined in clause 20, further comprising:
exposing a portion of the second photoresist to ultraviolet lightultraviolet (UV) light, thereby forming an insoluble layer on the surface of the insoluble support structure, whereby another portion of the second photoresist remains soluble; and
removing the soluble other portion of the second photoresist, thereby forming an aperture of each of the plurality of trans wells.

Clause 22. The method as defined in clause 21, further comprising:
forming a respective membrane over the aperture of at least some of the plurality of trans wells; and
inserting a nanopore in at least some of the respective membranes.

Clause 23. The method as defined in one of clauses 20-22, further comprising attaching a second substrate to the insoluble layer, thereby forming a cis well in fluid communication with each of the plurality of trans wells.

Clause 24. The method as defined in clause 23, further comprising securing a cis electrode in the second substrate prior to the attaching.

Clause 25. The method as defined in one of clauses 20-24, wherein:
a plurality of isolated electrodes is positioned on the substrate;
the first photoresist is deposited over each of the plurality of isolated electrodes and the substrate; and
each of the plurality of isolated electrodes is exposed when each of the plurality of trans wells is formed.

Clause 26. The method as defined in clause 25, wherein:
the plasma is an argon plasma when each of the plurality of isolated electrodes is silver/silver chloride; or
the plasma is an oxygen plasma when each of the plurality of isolated electrodes is a material other than silver/silver chloride.

Clause 27. A method, comprising:
depositing a photoresist over a substrate;
soft baking the photoresist;
exposing a portion of the photoresist to ultraviolet lightultraviolet (UV) light, thereby forming an insoluble support structure, whereby another portion of the photoresist remains soluble;
removing the soluble other portion of the photoresist, thereby forming a plurality of trans wells in the insoluble support structure; and
using reactive ion etching at a zero W substrate bias value to expose a top surface of the insoluble support structure to a directional fluorine-based plasma, thereby decreasing a surface energy of the top surface of insoluble support structure without decreasing the surface energy of sidewalls of the insoluble support structure that define the plurality of trans wells.

Clause 28. The method as defined in clause 27, wherein the fluorine-based plasma is selected from the group consisting of sulfur hexafluoride, tetrafluoromethane, and nitrogen trifluoride.

Clause 29. The method as defined in one of clauses 27-28, further comprising:
forming a respective membrane over a respective aperture of at least some of the plurality of trans wells; and
inserting a nanopore in at least some of the respective membranes.

Clause 30. The method as defined in one of clauses 27-29, wherein the each of the plurality of trans wells has a diameter of 50 μm or less.

Clause 31. The method as defined in one of clause 27-30, further comprising attaching a second substrate to the insoluble support structure, thereby forming a cis well in fluid communication with each of the plurality of trans wells.

Clause 32. The method as defined in clause 31, further comprising securing a cis electrode in the second substrate prior to the attaching.

Clause 33. The method as defined in one of clauses 27-32, wherein:
a plurality of isolated electrodes is positioned on the substrate;
the photoresist is deposited over each of the plurality of isolated electrodes and the substrate; and
each of the plurality of isolated electrodes is exposed when each of the plurality of trans wells is formed.

Clause 34. The method as defined in clause 33, wherein each of the plurality of isolated electrodes is selected from the group consisting of silver, silver chloride, and silver/silver chloride.

Clause 35. The method as defined in one of clauses 27-34, wherein an aspect ratio of each of the plurality of trans wells is 1:1 or more.

Clause 36. A method, comprising:
depositing a first photoresist over a substrate;
soft baking the first photoresist;
exposing the first photoresist to ultraviolet lightultraviolet (UV) light, thereby forming an insoluble first photoresist;
baking the insoluble first photoresist;
applying a fluoropolymer photoresist on the insoluble first photoresist;
soft baking the fluoropolymer photoresist;
using an etch mask and an oxygen plasma, selective etching through each of the fluoropolymer photoresist and the insoluble first photoresist to define a plurality of apertures in the fluoropolymer photoresist respectively leading to a plurality of trans wells in the insoluble first photoresist;
removing the etch mask; and
baking the insoluble first photoresist and the fluoropolymer photoresist, thereby lowering a surface energy of the fluoropolymer photoresist.

Clause 37. The method as defined in clause 36, further comprising:
forming a respective membrane over at least some of the plurality of apertures; and
inserting a nanopore in at least some of the respective membranes.

Clause 38. The method as defined in one of clauses 36-37, wherein the each of the plurality of trans wells has a diameter of 50 μm or less.

Clause 39. The method as defined in one of clauses 36-38, further comprising attaching a second substrate to fluoropolymer photoresist, thereby forming a cis well in fluid communication with each of the plurality of trans wells.

Clause 40. The method as defined in clause 39, further comprising securing a cis electrode in the second substrate prior to the attaching.

Clause 41. The method as defined in one of clauses 36-40, wherein:
a plurality of isolated electrodes is positioned on the substrate;
the first photoresist is deposited over each of the plurality of isolated electrodes and the substrate; and
each of the plurality of isolated electrodes is exposed when each of the plurality of trans wells is formed.

Clause 42. The method as defined in clause 41, wherein each of the plurality of isolated electrodes is selected from the group consisting of silver, silver chloride, and silver/silver chloride.

Clause 43. The method as defined in one of clauses 36-42, wherein an aspect ratio of each of the plurality of trans wells is 1:1 or more.

Clause 44. A method, comprising:
depositing a first photoresist over a substrate;
soft baking the first photoresist;
applying a fluoropolymer photoresist on the first photoresist;
soft baking the fluoropolymer photoresist;
using an etch mask, selective etching through the fluoropolymer photoresist to define a plurality of apertures in the fluoropolymer photoresist;
removing the etch mask;
exposing a portion of the first photoresist to ultraviolet lightultraviolet (UV) light, thereby forming an insoluble support structure, whereby other portions of the first photoresist that are adjacent to each of the plurality of apertures remains soluble;
removing the soluble other portions of the first photoresist, thereby forming a plurality of trans wells in the insoluble support structure that is respectively aligned with the plurality of apertures;
exposing the plurality of trans wells to an oxygen plasma, thereby increasing the surface energy of the insoluble support structure; and
baking the insoluble support structure and the fluoropolymer photoresist, thereby lowering a surface energy of the fluoropolymer photoresist.

Clause 45. The method as defined in clause 44, further comprising:
forming a respective membrane over at least some of the plurality of apertures; and
inserting a nanopore in the respective membrane.

Clause 46. The method as defined in one of clauses 44-45, wherein the each of the plurality of trans wells has a diameter of 50 μm or less.

Clause 47. The method as defined in one of clauses 44-46, further comprising attaching a second substrate to fluoropolymer photoresist, thereby forming a cis well in fluid communication with each of the plurality of trans wells.

Clause 48. The method as defined in clause 47, further comprising securing a cis electrode in the second substrate prior to the attaching.

Clause 49. The method as defined in one of clauses 44-48, wherein an aspect ratio of each of the plurality of trans wells is 1:1 or more.

### EXAMPLES

To further illustrate the present disclosure, examples are given herein. It is to be understood that these examples are provided for illustrative purposes and are not to be construed as limiting the scope of the present disclosure.

### NON-LIMITING WORKING EXAMPLES

### Example 1

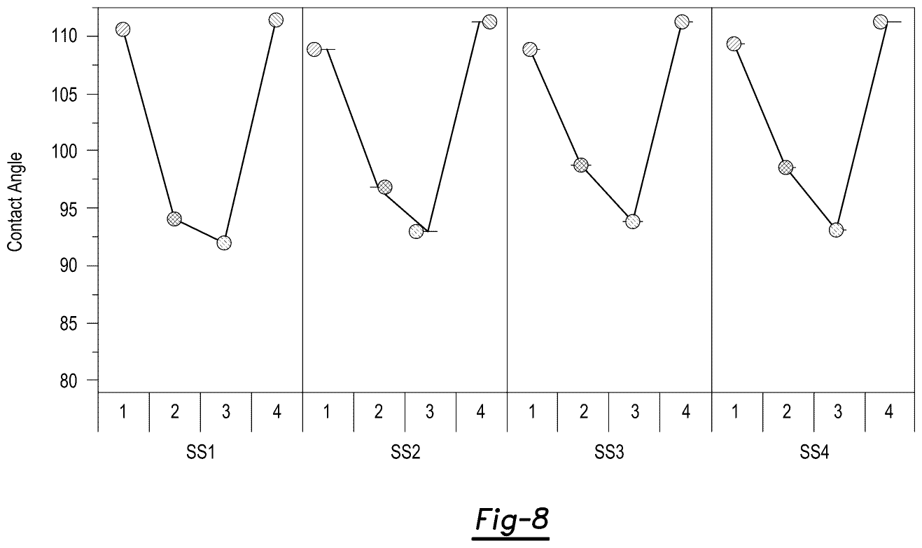

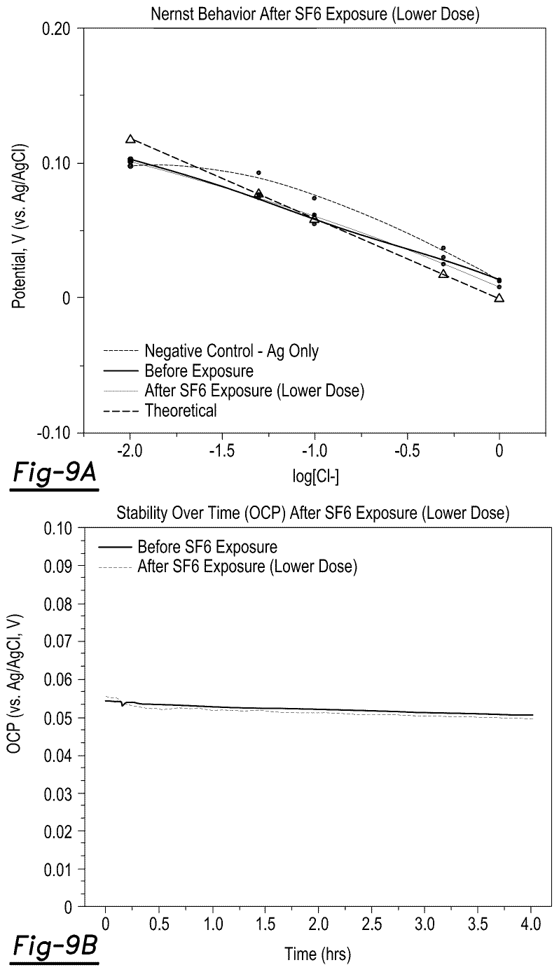

Four example support structures (SS1-SS4) were prepared via the method described in reference to FIG. 7A through FIG. 7E. The contact angle at the surface of the fluoropolymer photoresist was measured 1) initially post fabrication (i.e., after etching of the fluoropolymer photoresist and after UV light exposure/soluble portion removal of the underlying photoresist, 2) after O₂ plasma cleaning of the trans wells, 3) at a stage time to see temporal effect of the plasma, and 4) after an anneal step. The results are shown in FIG. 8, where the Y axis shows the contact angle (°) and the X axis identifies the support structure and the points at which the contact angle was measured (1-4 above). As shown, for each sample, O₂ plasma reduced the contact angle, and annealing restored the contact angle to >110°.

### Example 2

Optical inspection, wettability, and electrochemical testing in the form of Nernstian response and open circuit potential (OCP) were performed before and after exposure to hexafluoride (SF₆) to derisk the impact of plasma treatment on Ag/AgCl electrodes. The electrodes did not show meaningful changes in morphology or visual properties after exposure. The wells were still able to be wet with buffer using a suitable wetting process. As shown in FIG. 9A, the AgCl electrodes displayed Nernstian behavior after exposure with little deviation from pre-exposure behavior. As shown in FIG. 9B, a slight drift in OCP was observed but the signal was still stable and not considered to be a significant impact.

### Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A nanopore fluidic device, comprising:
a support structure having a top hydrophobic surface and having hydrophilic sidewalls defining a plurality of trans wells;
a trans electrode associated with each of the plurality of trans wells;
one or more cis wells facing the plurality of trans wells; and
a cis electrode associated with each of the one or more cis wells.

2. The nanopore fluidic device as defined in claim 1, further comprising respective membranes formed over at least some of the plurality of trans wells, each of the respective membranes being supported by the top hydrophobic surface.

3. The nanopore fluidic device as defined in claim 2, further comprising respective nanopores positioned in at least some of the respective membranes.

4. The nanopore fluidic device as defined in claim 1, wherein the top hydrophobic surface comprises a low surface energy plasma treatment selected from the group consisting of a fluorine plasma treatment and a tetramethyl silane.

5. (canceled)

6. The nanopore fluidic device as defined in claim 1, wherein the support structure comprises a top layer having the top hydrophobic surface and a sidewall layer having the hydrophilic sidewalls.

7. The nanopore fluidic device as defined in claim 6, wherein the top layer comprises a low surface energy material, and wherein the low surface energy material comprises a fluorine-containing material.

8. (canceled)

9. The nanopore fluidic device as defined in claim 6, wherein the sidewall layer comprises a high surface energy material.

10. The nanopore fluidic device as defined in claim 6, wherein the sidewall layer comprises a first photoresist and the top layer comprises a second photoresist layer.

11. The nanopore fluidic device as defined in claim 1, wherein the support structure comprises a photoresist.

12. The nanopore fluidic device as defined in claim 1, wherein each trans electrode is selected from the group consisting of Ag and AgCl.

13. The nanopore fluidic device as defined in claim 1, wherein:
i) each of the plurality of trans wells has a diameter of 50 μm or less; or
ii) an aspect ratio of each of the plurality of trans wells is 1:1 or more; or
iii) both i) and ii).

14. (canceled)

15. A method, comprising:
forming a material stack by:
depositing a first photoresist having a first surface energy over a substrate;
soft baking the first photoresist;
depositing a second photoresist having a second surface energy that is lower than the first surface energy over the first photoresist; and
soft baking the second photoresist;
exposing a portion of the material stack to ultraviolet light, thereby forming an insoluble support structure, whereby another portion of the material stack remains soluble; and
removing the soluble other portion, thereby forming a plurality of trans wells in the insoluble support structure.

16. The method as defined in claim 15, wherein an electrode is positioned on the substrate, and wherein the first photoresist is deposited over the electrode and the substrate.

17. The method as defined in claim 15, further comprising:
forming a respective membrane over a respective aperture of at least some of the plurality of trans wells; and
inserting a nanopore in at least some of the respective membranes.

18. The method as defined in claim 15, further comprising:
securing a cis electrode to the second substrate; and
then attaching a second substrate to the insoluble support structure, thereby forming a cis well in fluid communication with each of the plurality of trans wells.

19. (canceled)

20. A method, comprising:
depositing a first photoresist over a substrate;
soft baking the first photoresist;
exposing a portion of the first photoresist to ultraviolet light, thereby forming an insoluble support structure, whereby another portion of the first photoresist remains soluble;
removing the soluble other portion of the first photoresist, thereby forming a plurality of trans wells in the insoluble support structure;
exposing a surface of the insoluble support structure to a plasma, thereby increasing a surface energy of the insoluble support structure; and
laminating a second photoresist to the surface of the insoluble support structure.

21. The method as defined in claim 20, further comprising:
exposing a portion of the second photoresist to ultraviolet light, thereby forming an insoluble layer on the surface of the insoluble support structure, whereby another portion of the second photoresist remains soluble; and
removing the soluble other portion of the second photoresist, thereby forming an aperture of each of the plurality of trans wells.

22. The method as defined in claim 21, further comprising:
forming a respective membrane over the aperture of at least some of the plurality of trans wells; and
inserting a nanopore in at least some of the respective membranes.

23. The method as defined in claim 20, further comprising:
securing a cis electrode in the second substrate; and
then attaching a second substrate to the insoluble layer, thereby forming a cis well in fluid communication with each of the plurality of trans wells.

24. (canceled)

25. The method as defined in claim 20, wherein:
a plurality of isolated electrodes is positioned on the substrate;
the first photoresist is deposited over each of the plurality of isolated electrodes and the substrate; and
each of the plurality of isolated electrodes is exposed when each of the plurality of trans wells is formed.

26. The method as defined in claim 25, wherein:
the plasma is an argon plasma when each of the plurality of isolated electrodes is silver/silver chloride; or
the plasma is an oxygen plasma when each of the plurality of isolated electrodes is a material other than silver/silver chloride.

27.-49. (canceled)
